(19) United States
(12) Patent Application Publication
(10) Pub. No.: US 2011/0008267 A1
(43) Pub. Date: Jan. 13, 2011

(54) Pharmaceutical and Cosmeceutical Wash-Off Mousse Shampoo Compositions, Processes for Preparing the Same and Uses Thereof

(75) Inventors: Moshe Arkin, Kfar-Shemaryahu (IL); Amira Zeevi, Omer (IL); Nir Avram, Meitar (IL); Rina Uzan, Beer-Sheva (IL); Hagit Shilo-Volin, Meytar (IL); Erez Hollander, Arad (IL); Olga Buriakovsky, Beer-Sheva (IL)

Correspondence Address: THOMPSON HINE L.L.P.;Intellectual Property Group, P.O. BOX 8801, DAYTON, OH, 45401-8801, US

(73) Assignee: PERRIGO ISRAEL PHARMACEUTICALS LTD., Bnei-Brak (IL)

(21) Appl. No.: 12/887,021

(22) Filed: Sep. 21, 2010

Related U.S. Application Data
(62) Division of application No. 11/194,582, filed on Aug. 2, 2005.
(60) Provisional application No. 60/592,405, filed on Aug. 2, 2004.

(51) Int. Cl. A61K 9/12; A61P 17/10; A61P 17/12; A61P 29/00; A61P 17/14; A61P 17/00; A61P 31/04; A61P 17/02; A61P 35/00; A61P 31/10; A61P 17/06; A61P 33/10; A61P 33/00; A61P 31/00; A61P 17/08; A61P 31/12; A61P 9/08; A61P 17/18; A61P 7/10; A61P 25/24; A61P 17/04

(52) U.S. Cl. 424/43

(57) ABSTRACT

A method for treating a disease or disorder of the skin or scalp of a mammal while simultaneously cleansing the skin or scalp is disclosed. The method includes administering to the skin or scalp a mousse formed from a composition that includes a therapeutically or cosmeceutically effective amount of at least one active pharmaceutical ingredient, 10% to 50% by weight of a cleansing agent selected from the group consisting of anionic surfactants, nonionic surfactants and combinations thereof, a pharmaceutically acceptable mousse-forming carrier that includes a propellant, the propellant being 3% to 50% by weight of the composition, and water being about 40% to about 90% by weight of the composition, waiting a period of time; and rinsing said skin or scalp with water to remove the mousse.

### RELATED APPLICATIONS

This application is a divisional of U.S. Ser. No. 11/194,582 filed on Aug. 2, 2005, which claims priority from U.S. Ser. No. 60/592,405 filed on Aug. 2, 2004. The entire contents of the '582 and the '405 applications are incorporated herein by reference.

### FIELD

The present invention relates to the field of pharmacology and more particularly, to a wash-off mousse shampoo composition useful for topical delivery of active pharmaceutical ingredients to the scalp simultaneously with the cleaning of the hair, processes for the preparation thereof and methods of treatment using the same.

### BACKGROUND

There are many medical conditions that afflict the scalp. In addition to the discomfort caused by the condition itself, scalp conditions often cause an affected person great social discomfort. Further, the presence of hair on the scalp makes treatment of scalp conditions difficult, preventing or limiting an applied active pharmaceutical ingredient from making contact with an afflicted area. Additionally, the curved shape of the scalp and the impossibility of holding the head in a fixed position make application of active pharmaceutical ingredients to the scalp difficult. Further the proximity of the scalp to the eyes and mucous membranes often makes application difficult, unpleasant and often induces dread, especially when the treated person is a child.

Scalp conditions are generally treated with standard topical compositions, most often lotions, creams, pastes, gels, ointments, salves and milks, delivery forms developed and optimized for use in treating bare skin. These are generally unsuitable for application to hair as a large proportion of the composition sticks to the hair and does not contact the scalp.

Pharmaceutical compositions specifically formulated for delivering active pharmaceutical ingredients to the scalp are generally of one of three delivery forms: shampoos, solutions and sprays.

A solution pharmaceutical composition for the delivery of an active pharmaceutical ingredient to the scalp is often used. An amount of solution is poured on the head and quickly rubbed into the scalp with the fingers. It is generally difficult to apply a correct dose of an active pharmaceutical ingredient using a solution. A solution often drips away from the scalp, often to the eyes.

A spray pharmaceutical composition for the delivery of an active pharmaceutical ingredient to the scalp overcomes many of the problems associated with a solution pharmaceutical composition. The amount of composition applied to the hair is more easily regulated, making proper dosing relatively simple. There is reduced run-off and dripping when compared to a solution composition. Patient acceptance and compliance is generally good, especially with children. However, a spray pharmaceutical composition can easily enter the eyes. Further, self-application of a spray pharmaceutical composition is difficult.

Spray and solution compositions have a number of disadvantages in common. Both often leave unpleasant residue on the hands and fingers. Both require rubbing the composition into the scalp what may be uncomfortable to a person afflicted with a condition. Both are generally suitable only for the delivery of active pharmaceutical ingredients soluble in a single-phase solvent, limiting the type of active pharmaceutical ingredients that can be applied. Further, active pharmaceutical ingredients that are alcohol soluble may often not be used as the alcohol solvent often irritates or is harmful to a scalp afflicted with a condition.

A shampoo pharmaceutical composition is an effective general-purpose vehicle for the delivery of active pharmaceutical ingredients. Shampoos allow simultaneous cleansing of the hair with application of an active pharmaceutical ingredient, saving time and improving the quality of life of a person. Shampoos allow delivery of active pharmaceutical ingredients that are soluble in solvents other than water.

Shampoos have a number of disadvantages. Shampoos are typically and desirably applied on wet hair and thus oftentimes require wetting the hair prior to application. Usually it is necessary to rub a shampoo composition through the hair and onto the scalp, something that may be uncomfortable. Additionally, shampoo compositions have the tendency to drip and run into the eyes of a treated person, something that may be dangerous when the shampoo contains an active pharmaceutical ingredient. Further, it is difficult to apply an accurate dose of a shampoo pharmaceutical composition, leading to uneconomical use.

Mousses are a particularly convenient and pleasant-to-use product form for hair and scalp treatment formulations. The product is generally applied to the user's hand, where it forms a creamy foam which can be easily worked through the hair and scalp. Such mousses have found widespread use in the context of hair styling products. The conventional hair styling mousse generally utilizes a water soluble hair styling polymer, water, possibly a conditioning agent, an emulsifier, aesthetic agents and an aerosol propellant. The mousse is typically applied to hair dampened with water, spread through the hair and allowed to dry, giving a temporary set which can be removed by water or by shampooing.

Mousse-forming compositions (foamable compositions for application to the scalp) are generally single or multi-phase liquids provided in a pressurized container. When ejected from the pressurized container, the propellant expands, transforming the composition into a mousse.

Mousse shampoo compositions are known for the delivery of cleansing agents (e.g., U.S. Pat. No. 6,627,585). A mousse shampoo is applied to the head and spread over the hair. Since cleansing agents are oftentimes irritants if allowed to remain on the scalp for extended periods of time, the composition is subsequently washed out of the hair by rinsing with water.

Mousse-forming compositions are also described in U.S. Pat. No. 6,113,881. Such mousse-forming compositions are applied to the head usually after the hair has been cleaned with shampoo and left to dry so that the pharmaceutical or cosmetic ingredient remains on the scalp.

Mousse-forming compositions have many advantages over other compositions. The rigid yet fluid nature of a mousse allows a mousse-forming composition to be applied in any orientation without run-off as well as allowing convenient application of the mousse evenly over a large area. Mousses can be used dry, that is applied to hair that has not been wet with water. When applied onto damaged or sensitive skin, the mousse acts as a cushion, allowing spreading without direct physical contact. Mousse-forming compositions can be multiphase compositions so, unlike solutions, do not require that a component such as an active pharmaceutical ingredient be soluble in a specific solvent. Further, the fact that mousses can be multiphase compositions allows for the formulation of compositions containing various different beneficial ingredients. Desired or needed amounts of a mousse-forming composition can be accurately dispensed with ease, allowing for economical and efficient use. Due to these many advantages, mousse-forming compositions generally enjoy greater patient acceptance and compliance with treatment regimens.

The physical characteristics of a mousse formed by a mousse-forming composition are dependent upon the nature and relative amounts of components such as solvents, propellants and surface-active agents. Various mousse-forming compositions for the topical delivery of active pharmaceutical ingredients to the skin are taught, for example, in U.S. Pat. Nos. 3,856,956, 5,352,437, and 6,126,920.

Mousse-forming compositions that deliver both an active pharmaceutical ingredient and contain a cleansing agent are not known.

It would therefore be highly advantageous to have a convenient to use product for the delivery of active pharmaceutical ingredients that also allows simultaneous cleaning of the hair or any other bodily area.

### SUMMARY

The present invention successfully addresses the above-recited need by providing a pharmaceutical or cosmeceutical wash-off mousse shampoo composition containing, amongst other ingredients, at least one active pharmaceutical ingredient and at least one cleansing agent. For use, the composition of the present invention is applied to dry or wet hair and rubbed over the scalp. The active pharmaceutical ingredient comes in contact with the scalp while the cleansing agent cleans the hair. Optionally, the composition of the present invention is similarly applied on any dry or wet skin area that is afflicted by a skin disorder or disease, such that the active pharmaceutical ingredient comes in contact with the skin while the cleansing agent cleans the skin.

According to the teachings of the present invention there is provided a pharmaceutical or cosmeceutical wash-off mousse shampoo composition formulated for topical application to the skin and/or scalp of a mammal (human or non-human) comprising: (a) a cleansing agent; (b) a cosmeceutically or pharmaceutically effective amount of at least one active pharmaceutical ingredient; and (c) a pharmaceutically acceptable mousse-forming carrier.

In one embodiment, the wash-off mousse shampoo composition is formulated for application to dry hair. Wetting the hair, in this embodiment, is effected by the relatively large amount of water present in the composition (e.g., 70-80 weight percents).

In another embodiment, the wash-off mousse shampoo composition of the present invention is formulated for application to wet hair.

Typical cleansing agents used include but are not limited to cleansers, detergents and soaps.

Specific cleansing agents useful for implementing the teachings of the present invention include but are not limited to ammonium laureth sulfate, ammonium lauryl sulfate, ammonium myreth sulfate, “amphoteric-1”, “amphoteric-10”, “amphoteric-17”, “amphoteric-18”, “amphoteric-19”, “amphoteric-20”, “amphoteric-6”, coconut acid, saponified coconut oil, cocoyl sarcosine, DEA-laureth sulfate, DEA-lauryl sulfate, disodium monolauramido MEA-sulfo-succinate, disodium monococamido MIPA sulfosuccmate, disodium monococamidosulfo succinate, disodium monolaureth sulfo-succinate, disodium monooleamido MEA-sulfo-succinate, disodium monooleamidosulfo-succinate, disodium monoundecylenamido MEA sulfosuccinate, disodium monooleamido PEG-2 sulfosuccinate, dodecylbenzene sulfonic acid, isosteareth-6 carboxylic acid, lauroyl sarcosine, mixed isopropanolamines myristate, oleic acid, potassium cocoate, potassium coco-hydrolyzed animal protein, potassium cornate, sodium C12-C15 alcohols sulfate, sodium C14-C16 olefin sulfonate, sodium C16-C18 olefin sulfonate, sodium cocoglyceryl ether sulfonate, sodium cocoyl isothionate, sodium cocoyl sarcosinate, sodium dodecylbenzenesulfonate, sodium lauroyl sarcosinate, sodium lauryl sulfate, sodium myreth sulfate, sodium octoxynol-3 sulfonate, sodium/TEA-lauroyl hydrolyzed animal protein, TEA-cocoate, TEA-coco-hydrolyzed animal protein, TEA-dodecylbenzenesulfonate, TEA-laurate, TEA-lauryl sulfate, TEA-oleate, TEA-oleoyl sarcosinate, trideceth-7 carboxylic acid, potassium stearate, potassium undecylenoyl hydrolyzed animal protein and sodium laureth sulfate.

Substances which can also be used as cleansing agents according to the present invention are secondary degergents such as, but not limited to, ammonium nonoxynol4 sulfate, amphoteric-12, amphoteric-7, benzalkonium chloride, benzyl trimethyl ammonium hydrolyzed animal protein, capramide DEA, cocamide DEA, cocamide MEA, cocamidopropyl betaine, cocamidopropyl sultaine, cocamidopropylamine oxide, cocamine oxide, coco-betaine, DEA-lauraminopropionate, DEA-linoleate, dihydroxyethyl C12-C15 alkoxypropylamine oxide, dihydroxyethyl tallow glycinate, dioctyl sodium sulfosuccinate, fatty alkylolamide condensate, isostearamide DEA, lauramide DEA, lauramidopropyl betaine, lauramine oxide, laureth-12, laureth-23, lauryl betaine, linoleamide DEA, myristamide DEA, myristamine oxide, myristic acid, myristoyl hydrolyzed animal protein, nonoxynol-10, nonoxynol-12, nonoxynol-15, nonoxynol-9, octoxynol-13, octoxynol-9, oleamide DEA, oleamide mipa, oleth-20, oleth-3-phosphate, oleyl betaine, olive oil, palm kernelamide dea, peg-10 sorbitan laurate, peg-40 lanolin, peg-44 sorbitan laurate, peg-75 lanolin, peg-8 laurate, peg-85 lanolin, poloxamer 182, poloxamer 188, poloxamer 238, polysorbate 20, polysorbate 40, polysorbate 80, potassium ricinoleate, ppg-5-ceteth-10 phosphate, quaternium-20, quaternium-6, sodium cetyl sulfate, sodium lauraminopropionate, sodium laureth-12 sulfate, sodium lauroyl glutamate, sodium stearate, sodium tallow sulfate, soya acid, stearamide dea, stearic acid, sucrose cocoate, sulfated castor oil, oleth-10 phosphate, PEG-6 cocamide and sodium lauriminodipropionate.

Generally, the wash-off mousse shampoo composition of the present invention may include irritant ingredients. Thus, in an embodiment of the present invention the composition is formulated so that the composition can be washed out of the area to which the composition is applied (e.g., hair) within 20 minutes, within 10 minutes or even within 5 minutes of application. In any event, anti-irritants can be added to the compositions, so as to minimize or abolish any irritation that may be caused while the composition remains on the treated area.

According to a feature of the present invention, the concentration of the at least one active pharmaceutical ingredient in the composition is high enough so as to allow a sufficient amount of the active pharmaceutical ingredient to come in contact with the skin, before being washed off. Depending on the nature of the active pharmaceutical ingredient and the other composition components, in some embodiments the concentration of the at least one active pharmaceutical ingredient is at least about 0.01 weight percentages, at least about 0.1 weight percentages, at least about 1 weight percentage and even at least about 3 weight percentages, of the total weight of the composition.

Preferably, at least one of the at least one active pharmaceutical ingredients in the composition is for the treatment of skin and/or scalp diseases and disorders. Such active pharmaceutical ingredients useful in implementing the teachings of the present invention include but are not limited to active herbal extracts, acaricides, age spot and keratose removing agents, analgesics, local anesthetics, antiacne agents, antiaging agents, antibacterials, antibiotics, antiburn agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antioxidants, antipruritics, antipsoriatic agents, antirosacea agents, antiseborrheic agents, antiseptic, antiswelling agents, antiviral agents, antiyeast agents, astringents, topical cardiovascular agents, chemotherapeutic agents, corticosteroids, fungicides, hair growth regulators, hormones, hydroxyacids, insecticides, keratolytic agents, lactams, mitocides, non-steroidal anti-inflammatory agents, pediculicides, progestins, sanatives, scabicides, vasodilators and wart removers.

In an embodiment of the present invention, the pharmaceutically acceptable mousse-forming carrier includes a propellant. Suitable propellants include but are not limited to nitrous oxide, carbon dioxide, chloropentafluoroethane, dichlorodifluoromethane, nitrogen, propane, iso-butane, n-butane, isopentane, n-pentane, dimethyl ether, trichlorofluoromethane and mixtures thereof. Generally, a propellant makes up from about 3 weight percents to about 50 weight percents of the total weight of the composition.

In an embodiment of the present invention, the pharmaceutically acceptable mousse-forming carrier further comprises a foam-forming agent, which typically includes one or more surface-active agents. The concentration of the surface-active agents in a composition of the present invention is generally between about 0.1 weight percent and about 50 weight percents of the total weight of the composition, and, more preferably is between about 5 weight percents and about 50 weight percents of the total weight of the composition, more preferably is between about 10 weight percents and about 50 weight percents of the total weight of the composition, more preferably is between about 15 weight percents and about 50 weight percents of the total weight of the composition, and even more preferably is between about 20 weight percents and about 50 weight percents of the total weight of the composition.

In another embodiment of the present invention, the mousse-forming carrier further comprises at least one additional component selected from the group consisting of emulsifiers, fatty alcohols (e.g., ethoxylated fatty alcohols), hydrocarbon alcohols and water.

In an embodiment of the present invention, a wash-off mousse shampoo composition of the present invention includes one or more additional ingredients. Such additional ingredients include but are not limited to anti perspirants, anti-static agents, buffering agents, bulking agents, chelating agents, colorants, conditioners, deodorants, diluents, dyes, emollients, fragrances, hair conditioners, humectants, occlusive agents, oils, penetration enhancers, pearlescent aids, perfuming agents, permeation enhancers, pH-adjusting agents, preservatives, protectants, skin penetration enhancers, softeners, solubilizers, sunscreens, sun blocking agents, sunless tanning agents, viscosity modifiers and vitamins. As is known to one skilled in the art, in some instances a specific additional component may have more than one activity, function or effect.

In an embodiment of the present invention, the wash-off mousse shampoo composition described herein is packaged in a packaging material and identified in print, in or on the packaging material, for use for a need selected from the group consisting of curing a condition, treating a condition, preventing a condition, treating symptoms of a condition, curing symptoms of a condition, ameliorating symptoms of a condition, treating effects of a condition, ameliorating effects of a condition, and preventing results of a condition.

In an embodiment of the present invention, the condition is selected from the group consisting of a medical condition and a cosmeceutical condition, especially a skin and/or scalp disease or disorder. Typical such skin and/or scalp disease or disorders include acne rosacea, actinic keratoses, actinic porokeratosis, acute inflammatory diseases, age spots, allergic contact dermatitis, alopecia, asteatotic eczema, atopic dermatitis, atopic eczema, bacterial infection, BCC, Bowen's disease, burns, chronic hypertrophic lichen planus, chronic superficial scaling, contact dermatitis, cradle cap, cutaneous T-cell lymphoma, cystic acne, dandruff, Darier's disease, dermatitis, dermatitis herpetiformis, dermatosis, discoid eczema, discoid lupus erythematosus, dry skin, eczema, erythrasma, exfoliative keratolysis, folliculitis, fungal infection, juvenile plantar dermatosis, granuloma annulare, Grover's disease, hair thinning, ichthyosiform dermatoses, ichthyosis, impetigo, infantile eczema, infection, intertrigo, keratosis, keloid scarsm lichen simplex chronicus, lichen planus, lichen striatus, lupus erythematosus, neurodermatitis, palmar hyperkeratosis, palmoplantar psoriasis, papular urticaria, parapsoriasis, pediculosis, pellagra, perifolliculitis, pigmented skin, lesions, pityriasis alba, pityriasis lichenoides, pityriasis rosea, pityriasis rubra pilaris, pityriasis versicolor, plantar hyperkeratosis, neurodermatitis, pruritis, psoriasis, Reiter's syndrome, rosacea, seborrhoeic dermatitis, subacute cutaneous lupus erythematosus, tinea capitis, superficial BCC, warts, wound, wrinkles and yeast infections (especially of Malassezia ovalis, Malassezia furfur, Pityrosporium orbiculare and Pityrosporium ovale).

According to the teachings of the present invention, there is also provided a process for preparing a pharmaceutical or cosmeceutical wash-off mousse shampoo composition of the present invention by: (a) obtaining a mixture of a cleansing agent, at least one active pharmaceutical ingredient and a pharmaceutically acceptable mousse-forming carrier; (b) placing the mixture in a pressure-resistant vessel; and (c) sealing the pressure-resistant vessel.

In a preferred embodiment of this aspect of the present invention, the above described mixture, excluding the propellant, is placed in a pressure-resistant vessel and the vessel is fitted with a seal-valve. The propellant is then placed in the pressure-resistant vessel, and the seal-valve of the pressure-resistant vessel is fitted with an actuator.

Suitable cleansing agents include but are not limited to cleansers, detergents and soaps.

According to the teachings of the present invention there is also provided a method of treating a skin and/or scalp disease or disorder, the method involving (a) topically applying to a skin and/or scalp area afflicted by the disease or disorder of a mammal (human or non-human) in need thereof a therapeutically or cosmeceutically effective amount of at least one active pharmaceutical ingredient simultaneously with a cleansing agent in a mousse form; (b) subsequent to the applying, waiting a period of time, and (c) subsequent to waiting, rinsing the skin and/or scalp area.

According to a feature of the present invention, the need for which the method of the present invention is applied includes curing a condition, treating a condition, preventing a condition, treating symptoms of a condition, curing symptoms of a condition, ameliorating symptoms of a condition, treating effects of a condition, ameliorating effects of a condition, and preventing results of a condition. Conditions include medical conditions and cosmeceutical conditions, such as skin and/or scalp diseases and disorders, as is detailed hereinabove.

The specific active pharmaceutical ingredient or ingredients administered is dependent on the need for which the method is implemented. Suitable active pharmaceutical ingredients include but are not limited to active herbal extracts, acaricide, age spots and keratoses removing agents, analgesics, local anesthetics, antiacne agents, antiaging agents, antibacterials, antibiotics, antiburn agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antioxidants, antipruritics, agents, antipsoriatic agents, antirosacea, antiseborrheic agents, antiseptic, antiswelling agents, antiviral agents, antiyeast agents, astringents, topical cardiovascular agents, chemotherapeutics, corticosteroids, fungicides, hair growth regulators, hormones, hydroxyacids, insecticides, keratolytics, lactams, mitocides, non-steroidal antiinflammatory agents, pediculicide, progestins, sanatives, scabicide, vasodilators and wart removers. As is known to one skilled in the art, in some instances a specific active pharmaceutical ingredient may have more than one activity, function or effect.

In one embodiment of the present invention, the skin and/or scalp area is wet before applying the active pharmaceutical ingredient and the cleansing agent. In another embodiment of the present invention, the skin and/or scalp area is not wet before applying the active pharmaceutical ingredient and the cleansing agent.

The skin and/or scalp area is preferably a hirsute area such as the hair.

Depending on the embodiment, the period of time waited can be less than 20 minutes, less than 10 minutes or even less than 5 minutes. Consequently, the cosmeceutically or pharmaceutically effective amount of the active pharmaceutical ingredient administered must be high enough so as to allow the absorption of a sufficient amount of the active pharmaceutical ingredient into the skin or scalp, or to allow the active pharmaceutical ingredient to exert its activity in that period of time.

In an embodiment of the present invention, topically applying simultaneously the active pharmaceutical ingredient and the cleansing agent is effected while utilizing a wash-off mousse shampoo composition that comprises the active pharmaceutical ingredient and the cleansing agent. The composition is preferably applied by passing a pharmaceutical or cosmeceutical wash-off mousse shampoo composition including the at least one active pharmaceutical ingredient, the cleansing agent and a mousse forming-carrier from a first volume (e.g. a vessel) having a first pressure through a passage (e.g. a nozzle) into a second volume (e.g. the open air) having a second pressure, the first pressure being greater than the second pressure, so as to effect foaming of the composition. A preferred wash-off mousse shampoo composition for implementing the method of the present invention is the wash-off mousse shampoo composition of the present invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents and other references mentioned herein are incorporated by reference in their entirety. In case of conflict, the patent specification, including definitions, will control. In addition, the materials, methods, and examples are illustrative only and not intended to be limiting.

### DETAILED DESCRIPTION

The present invention is of a pharmaceutical or cosmeceutical wash-off mousse shampoo composition containing at least one active pharmaceutical ingredient that is useful for the topical delivery of the at least one active pharmaceutical ingredients to hair or skin of a mammal, whether a human or non-human mammal, simultaneously with the delivery of a cleansing agent to the hair or skin. The present invention also includes a process for the preparation of the wash-off mousse shampoo composition of the present invention. The present invention also includes methods of treatments of skin, especially of the scalp, by the simultaneous delivery of an active pharmaceutical ingredient and a cleansing agent in a mousse form, followed by rinsing of the area.

As discussed hereinabove, a mousse delivery form has many advantages for the topical dispensation of active pharmaceutical ingredients by providing quick and accurate dosage, high penetration, convenient application, ease of application, economy in use, and increased safety by avoiding contact of composition components with mucous membranes and the eyes to large areas of the scalp surface. Further, mousses for application to the scalp have greater acceptability and subsequently compliance than other delivery forms, especially amongst children.

The principles, uses and implementations of the present invention are better understood with reference to the accompanying descriptions and examples.

Before explaining at least one embodiment of the invention in detail, it is to be understood that the invention is not limited in its application to the details set forth herein. The invention can be implemented with other embodiments and can be practiced or carried out in various ways. It is also understood that the phraseology and terminology employed herein is for descriptive purpose and should not be regarded as limiting.

As used herein, the term “comprising” means that other steps and ingredients which do not affect the final result can be added. This term encompasses the terms “consisting of” and “consisting essentially of”.

The phrase “consisting essentially of” means that the composition may include additional ingredients, but only if the additional ingredients do not materially alter the basic and novel characteristics of the claimed compositions or methods.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “pharmaceutical composition” refers to any composition, which contains at least one active pharmaceutical ingredient and is suitable for administration to a patient.

By “topical administration” or “topical application” is meant the application of a therapeutically effective amount of a pharmaceutical composition to the external and/or exposed surface of the skin, to access the dermis and/or epidermis.

By “therapeutically effective amount” is meant an amount sufficient to provide some medical, cosmeceutical or cosmetic benefit.

The term “active pharmaceutical ingredient” refers to a pharmaceutical or cosmeceutical agent including any natural or synthetic chemical substance that subsequent to being applied to a mammal has at least one desired pharmaceutical effect.

The term “topical active pharmaceutical ingredient” refers to a pharmaceutical or cosmeceutical agent including any natural or synthetic chemical substance, intended for topical application on a surface of a mammal, especially to the skin, and that subsequent to the topical application has at least one desired pharmaceutical effect.

The pharmaceutical or cosmeceutical wash-off mousse shampoo composition of the present invention substantially comprises (a) a cleansing agent, (b) a cosmeceutically or pharmaceutically effective amount of at least one active pharmaceutical ingredient and (c) a pharmaceutically acceptable mousse-forming carrier. The wash-off mousse composition of the present invention can be formulated to be applicable to either or both wet hair or dry hair. When applied on dry hair, wetting the hair is effected by water, which is present in a relatively large amount in the formulation.

Typical cleansing agents used include but are not limited to cleansers, detergents and soaps. Specific cleansing agents useful for implementing the teachings of the present invention include but are not limited to ammonium laureth sulfate, ammonium lauryl sulfate, ammonium myreth sulfate, “amphoteric-1”, “amphoteric-10”, “amphoteric-17”, “amphoteric-18”, “amphoteric-19”, “amphoteric-20”, “amphoteric-6”, coconut acid, saponified coconut oil, cocoyl sarcosine, DEA-laureth sulfate, DEA-lauryl sulfate, disodium monolauramido MEA-sulfo-succinate, disodium monococamido MIPA sulfosuccmate, disodium monococamidosulfo succinate, disodium monolaureth sulfo-succinate, disodium monooleamido MEA-sulfo-succinate, disodium monooleamidosulfo-succinate, disodium monoundecylenamido MEA sulfosuccinate, disodium monooleamido PEG-2 sulfosuccinate, dodecylbenzene sulfonic acid, isosteareth-6 carboxylic acid, lauroyl sarcosine, mixed isopropanolamines myristate, oleic acid, potassium cocoate, potassium coco-hydrolyzed animal protein, potassium comate, sodium C12-C15 alcohols sulfate, sodium C14-C16 olefin sulfonate, sodium C16-C18 olefin sulfonate, sodium cocoglyceryl ether sulfonate, sodium cocoyl isothionate, sodium cocoyl sarcosinate, sodium dodecylbenzenesulfonate, sodium lauroyl sarcosinate, sodium lauryl sulfate, sodium myreth sulfate, sodium octoxynol-3 sulfonate, sodium/TEA-lauroyl hydrolyzed animal protein, TEA-cocoate, TEA-coco-hydrolyzed animal protein, TEA-dodecylbenzenesulfonate, TEA-laurate, TEA-lauryl sulfate, TEA-oleate, TEA-oleoyl sarcosinate, trideceth-7 carboxylic acid, potassium stearate, potassium undecylenoyl hydrolyzed animal protein and sodium laureth sulfate.

Substances which can also be used as cleansing agents according to the present invention are secondary degergents such as, but not limited to, ammonium nonoxynol-4 sulfate, amphoteric-12, amphoteric-7, benzalkonium chloride, benzyl trimethyl ammonium hydrolyzed animal protein, capramide DEA, cocamide DEA, cocamide MEA, cocamidopropyl betaine, cocamidopropyl sultaine, cocamidopropylamine oxide, cocamine oxide, coco-betaine, DEA-lauraminopropionate, DEA-linoleate, dihydroxyethyl C12-C15 alkoxypropylamine oxide, dihydroxyethyl tallow glycinate, dioctyl sodium sulfosuccinate, fatty alkylolamide condensate, isostearamide DEA, lauramide DEA, lauramidopropyl betaine, lauramine oxide, laureth-12, laureth-23, lauryl betaine, linoleamide DEA, myristamide DEA, myristamine oxide, myristic acid, myristoyl hydrolyzed animal protein, nonoxynol-10, nonoxynol-12, nonoxynol-15, nonoxynol-9, octoxynol-13, octoxynol-9, oleamide DEA, oleamide mipa, oleth-20, oleth-3 phosphate, oleyl betaine, olive oil, palm kernelamide dea, peg-10 sorbitan laurate, peg-40 lanolin, peg-44 sorbitan laurate, peg-75 lanolin, peg-8 laurate, peg-85 lanolin, poloxamer 182, poloxamer 188, poloxamer 238, polysorbate 20, polysorbate 40, polysorbate 80, potassium ricinoleate, ppg-5-ceteth-10 phosphate, quaternium-20, quaternium-6, sodium cetyl sulfate, sodium lauraminopropionate, sodium laureth-12 sulfate, sodium lauroyl glutamate, sodium stearate, sodium tallow sulfate, soya acid, stearamide dea, stearic acid, sucrose cocoate, sulfated castor oil, oleth-10phosphate, PEG-6 cocamide and sodium lauriminodipropionate.

Active pharmaceutical ingredients that can be advantageously delivered using the teachings of the present invention include topical active pharmaceutical ingredient for the treatment of skin and/or scalp diseases and disorders. Such active pharmaceutical ingredients include but are not limited to active herbal extracts, acaricides, age spot and keratose removing agents, analgesics, local anesthetics, antiacne agents, antiaging agents, antibacterials, antibiotics, antiburn agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antioxidants, antipruritics, antipsoriatic agents, antirosacea agents, antiseborrheic agents, antiseptics, antiswelling agents, antiviral agents, antiyeast agents, astringents, topical cardiovascular agents, chemotherapeutic agents, corticosteroids, fungicides, hair growth regulators, hormones, hydroxyacids, insecticides, keratolytic agents, lactams, mitocides, non-steroidal anti-inflammatory agents, pediculicides, progestins, sanatives, scabicides, vasodilators and wart removers. It is important to note that in some cases a specific active pharmaceutical ingredient has one or more types of activities and therefore falls within more than one of the types listed above.

Suitable active herbal extracts include, but are not limited to angelica, anise oil, astragali radix, azalea, benzyl acetate, birch tar oil, bornyl acetate, cacumen biotae, calendula, camphor, cantharidin, capsicum, chamomile, cineole, cinnamon bark, cinnamon leaf, citronella, citronellol, citronellyl acetate, citronellyl formate, eucalyptus, eugenyl acetate, fennel, flos carthami, fructus mori, garlic, geraniol, geranium, geranyl acetate, grape, habanera, horsetail, isobutyl angelicate, jojoba, lavender, ledum latifolium, ledum palustre, lemongrass, licorice, limonene, linalool, linalyl acetate, methyl anthranilate, methyl cinnamate, mezereum, neem, nerol, neryl acetate, nettle root extract, oleum ricini, oregano, pinenes, .alpha.-pinene, .beta.-pinene, radix angelicae sinesis, radix paenoiae rubra, radix polygoni multiflori, radix rehmanniae, rhizoma pinelliae, rhizoma zingiberis recens, rosemary, sabadilla, sage, sandalwood oil, saw palmetto extract, semen sesami nigrum, shea butter, staphysagria, tea tree oil, terpene alcohols, terpene hydrocarbons, terpene esters, terpinene, terpineol, terpinyl acetate, and derivatives, esters, salts and mixtures thereof.

Suitable acaricides include, but are not limited to amitraz, flumethrin, fluvalinate, and derivatives, esters, salts and mixtures thereof.

Suitable age spots and keratoses removing agents include, but are not limited to hydroxyacids, hydroquinone, and derivatives, esters, salts and mixtures thereof.

As is known to one skilled in the art, many agents have analgesic and/or anesthetic and/or antipruritic activity. Suitable analgesics include but are not limited to benzocaine, butamben picrate, dibucaine, dimethisoquin, dyclonine, lidocaine, pramoxine, tetracaine, salicylates and derivatives, esters, salts and mixtures thereof. Suitable local anesthetics include but are not limited to benzocaine, bupivacaine, butamben picrate, chlorprocaine, cocaine, dibucaine, dimethisoquin, dyclonine, etidocaine, hexylcaine, ketamine, lidocaine, mepivacaine, pramoxine, procaine, tetracaine, salicylates and derivatives, esters, salts and mixtures thereof. Suitable antipruritics include, but are not limited to menthol, methdilazine, trimeprazine, urea and derivatives, esters, salts and mixtures thereof.

Suitable antiacne agents include, but are not limited to N-acetylcysteine, adapalene, azelaic acid, benzoyl peroxide, cholate, clindamycin, deoxycholate, erythromycin, flavinoids, glycolic acid, meclocycline, mupirocin, octopirox, phenoxy ethanol, phenoxy proponol, pyruvic acid, resorcinol, retinoic acid, salicylic acid, scymnol sulfate, sulfacetamide-sulfur, sulfur, tazarotene, tetracycline, tretinoin triclosan, and derivatives, esters, salts and mixtures thereof.

Suitable antiaging agents include, but are not limited to melatonin, and derivatives, esters, salts and mixtures thereof.

As is known to one skilled in the art, the term antibiotic includes agents with antimicrobial, antibacterial, antimycotic, antiprotoazaol activity. Suitable antibiotics include, but are not limited to amanfadine hydrochloride, amanfadine sulfate, amikacin, amikacin sulfate, aminoglycosides, amoxicillin, ampicillin, ansamycins, azelaic acid, bacitracin, beta-lactams, candicidin, capreomycin, carbenicillin, cephalexin, cephaloridine, cephalothin, cefazolin, cephapirin, cephradine, cephaloglycin, chloramphenicols, chlorhexidine, chlorhexidine gluconate, chlorhexidine hydrochloride, chloroxine, chlorquinaldol, chlortetracycline, chlortetracycline hydrochloride, ciprofloxacin, circulin, clindamycin, clindamycin hydrochloride, clotrimazole, cloxacillin, demeclocycline, diclosxacillin, diiodohydroxyquin, doxycycline, ethambutol, ethambutol hydrochloride, erythromycin, erythromycin estolate, erythromycin stearate, farnesol, floxacillin, gentamicin, gentamicin sulfate, gramicidin, griseofulvin, haloprogin, haloquinol, hexachlorophene, iminocylcline, iodochlorhydroxyquin, kanamycin, kanamycin sulfate, lincomycin, lineomycin, lineomycin hydrochloride, macrolides, meclocycline, methacycline, methacycline hydrochloride, methenamine, methenamine hippurate, methenamine mandelate, methicillin, metronidazole, metronidazole hydrochloride, miconazole, miconazole hydrochloride, minocycline, minocycline hydrochloride, mupirocin, nafcillin, neomycin, neomycin sulfate, netilmicin, netilmicin sulfate, nitrofurazone, norfloxacin, nystatin, octopirox, oleandomycin, orcephalosporins, oxacillin, oxytetracycline, oxytetracycline hydrochloride, parachlorometa xylenol, paromomycin, paromomycin sulfate, penicillins, penicillin G, penicillin V, pentamidine, pentamidine hydrochloride, phenethicillin, polymyxins, quinolones, streptomycin sulfate, tetracycline, tobramycin, tolnaftate, triclosan, trifampin, rifamycin, rolitetracycline, spectinomycin, spiramycin, streptomycin, sulfonamide, tetracyclines, tetracycline, tobramycin, tobramycin sulfate, triclocarbon, triclosan, trimethoprim-sulfamethoxazole, tylosin, vancomycin, yrothricin and derivatives, esters, salts and mixtures thereof.

Specifically, suitable antimycotics include, but are not limited to azole compounds, chloroxine, ciclopirox olamine, clotrimazole, econazole, fluconazole, griseofulvin, mafenide acetate, nystatin, terbinafine, undecylenic acid, and derivatives, esters, salts and mixtures thereof.

Suitable antidandruff agents include, but are not limited to aminexil, benzalkonium chloride, benzethonium chloride, 3-bromo-1-chloro-5,5-dimethyl-hydantoin, chloramine B, chloramine T, chlorhexidine, N-chlorosuccinimide,climbazole, 1,3-dibromo-5,5-dimethylhydantoin, 1,3-dichloro-5,5-dimethyl-hydantoin, betulinic acid, betulonic acid, celastrol, crataegolic acid, cromakalin, cyproterone acetate, dutasteride, finesteride, ibuprofen, ketoconozole, oleanolic acid, phenyloin, picrotone olamine, salicylic acid, selenium sulphides, triclosan, triiodothyronine, ursolic acid, zinc gluconate, zinc omadine, zinc pyrithione, and derivatives, esters, salts and mixtures thereof.

Suitable antidepressants include, but are not limited to norepinephrine-reuptake inhibitors, selective-serotonin-reuptake inhibitors, monoamine-oxidase inhibitors, serotonin-and-noradrenaline-reuptake inhibitors, corticotropin-releasing factor antagonists, .alpha.-adrenoreceptor antagonists, NK1-receptor antagonists, 5-HT.sub.1A-receptor agonist, antagonists, amitriptyline, desmethylamitriptyline, clomipramine, doxepin, imipramine, imipramine-oxide, trimipramine, adinazolam, amiltriptylinoxide, amoxapine, desipramine, maprotiline, nortriptyline, protriptyline, amineptine, butriptyline, demexiptiline, dibenzepin, dimetacrine, dothiepin, fluacizine, iprindole, lofepramine, melitracen, metapramine, norclolipramine, noxiptilin, opipramol, perlapine, pizotyline, propizepine, quinupramine, reboxetine, tianeptine, binedaline, m-chloropiperzine, citalopram, duloxetine, etoperidone, femoxetine, fluoxetine, fluvoxamine, indalpine, indeloxazine, milnacipran, nefazodone, oxaflazone, paroxetine, prolintane, ritanserin, sertraline, tandospirone, venlafaxine and zimeldine, and derivatives, esters, salts and mixtures thereof.

Suitable antihistamines include, but are not limited to chlorcyclizine, diphenhydramine, mepyramine, methapyrilene, tripelennamine and derivatives, esters, salts and mixtures thereof.

Suitable antipsoriatic agents include, but are not limited to 6-aminonicotinamide, 6-aminonicotinic acid, 2-aminopyrazinamide, anthralin, calcipotriene, 6-carbamoylnicotinamide, 6-chloronicotinamide, 2-carbamoylpyrazinamide, corticosteroids, 6-dimethylaminonicotinamide, dithranol, 6-formylaminonicotinamide, 6-hydroxy nicotinic acid, 6-substituted nicotinamides, 6-substituted nicotinic acid, 2-substituted pyrazinamide, tazarotene, thionicotinamide, trichothecene mycotoxins, and derivatives, esters, salts and mixtures thereof.

Suitable antirosacea agents include, but are not limited to azelaic acid, metronidazole sulfacetamide, and derivatives, esters, salts and mixtures thereof.

Suitable antiseborrheic agents include, but are not limited to glycolic acid, salicylic acid, selenium sulfide, zinc pyrithione and derivatives, esters, salts and mixtures thereof.

Suitable antiviral agents include, but are not limited to acyclovir, and derivatives, esters, salts and mixtures thereof.

Suitable chemotherapeutic agents include, but are not limited to daunorubicin, doxorubicin, lomustine, fotemustine, idarubicin, amrubicin, pirarubicin, epirubicin, mitoxantrone, etoposide, teniposide, vinblastine, vincristine, mitomycin C, 5-FU, paclitaxel, docetaxel, actinomycin D, colchicine, topotecan, irinotecan, gemcitabine cyclosporin, verapamil, valspodor, probenecid, MK571, GF120918, LY335979, biricodar, terfenadine, quinidine, pervilleine A, XR9576, and derivatives, esters, salts and mixtures thereof.

Suitable corticosteroids include, but are not limited to alclometasone dipropionate, amcinafel, amcinafide, amcinonide, beclomethasone, beclomethasone dipropionate, betamethsone, betamethasone benzoate, betamethasone dexamethasonephosphate, dipropionate, betamethasone valerate, budesonide, chloroprednisone, chlorprednisone acetate, clescinolone, clobetasol, clobetasol propionate, clobetasol valerate, clobetasone, clobetasone butyrate, clocortelone, cortisone, cortodoxone, craposone butyrate, desonide, desoxymethasone, dexamethasone, desoxycorticosterone acetate, dichlorisone, diflorasone diacetate, diflucortolone valerate, difluorosone diacetate, diflurprednate, fluadrenolone, flucetonide, flucloronide, fluclorolone acetonide, flucortine butylesters, fludroxycortide, fludrocortisone, flumethasone, flumethasone pivalate, flumethasone pivalate, flunisolide, fluocinolone, fluocinolone acetonide, fluocinonide, fluocortin butyl, fluocortolone, fluorometholone, fluosinolone acetonide, fluperolone, fluprednidene acetate, fluprednisolone hydrocortamate, fluradrenolone, fluradrenolone acetonide, flurandrenolone, fluticasone, halcinonide, hydrocortisone, hydrocortisone acetate, hydrocortisone butyrate, hydrocortisone cyclopentylpropionate, hydrocortisone valerate, hydroxyltriamcinolone, medrysone, meprednisone, .alpha.-methyl dexamethasone, methylprednisolone, methylprednisolone acetate, mometasone furoate, paramethasone, prednisolone, prednisone, pregnenolone, progesterone, spironolactone, triamcinolone, triamcinolone acetonide, and derivatives, esters, salts and mixtures thereof.

Suitable hair growth regulators include, but are not limited to N-acetylgalactosamine, N-acetylglucosamine, N-acetylmannosamine, acitretin, aminexil, ascomycin, asiatic acid, azelaic acid, benzalkonium chloride, benzethonium chloride, benzydamine, benzyl nicotinate, benzoyl peroxide, benzyl peroxide, betulinic acid, betulonic acid, calcium pantothenate, celastrol, cepharanthine, chlorpheniramine maleate, clinacycin hydrochloride, crataegolic acid, cromakalin, cyproterone acetate, diazoxide, diphenhydramine hydrochloride, dutasteride, estradiol, ethyl-2-hydroxypropanoate, finasteride, D-fucono-1,5-lactone, furoate, L-galactono-1,4-lactone, D-galactosamine, D-glucaro-1,4-lactone, D-glucosamine-3-sulphate, hinokitiol, hydrocortisone, 2-hydroxypropionic acid, isotretinoin, itraconazole, ketoconazole, latanoprost, 2-methyl propan-2-ol, minocyclin, minoxidil, mipirocin, mometasone, oleanolic acid, panthenol, 1,10-phenanthroline, phenyloin, prednisolone, progesterone, propan-2-ol, pseudoterins, resorcinol, selenium sulfide, tazarotene, triclocarbon, triclosan, triiodothyronine, ursolic acid, zinc pyrithione, and derivatives, esters, salts and mixtures thereof.

Suitable hormones include, but are not limited to methyltestosterone, androsterone, androsterone acetate, androsterone propionate, androsterone benzoate, androsteronediol, androsteronediol-3-acetate, androsteronediol-17-acetate, androsteronediol 3-17-diacetate, androsteronediol-17-benzoate, androsteronedione, androstenedione, androstenediol, dehydroepiandrosterone, sodium dehydroepiandrosterone sulfate, dromostanolone, dromostanolone propionate, ethylestrenol, fluoxymesterone, nandrolone phenpropionate, nandrolone decanoate, nandrolone furylpropionate, nandrolone cyclohexane-propionate, nandrolone benzoate, nandrolone cyclohexanecarboxylate, androsteronediol-3-acetate-1-7-benzoate, oxandrolone, oxymetholone, stanozolol, testosterone, testosterone decanoate, 4-dihydrotestosterone, 5.alpha.-dihydrotestosterone, testolactone, 17.alpha.-methyl-19-nortestosterone, desogestrel, dydrogesterone, ethynodiol diacetate, medroxyprogesterone, levonorgestrel, medroxyprogesterone acetate, hydroxyprogesterone caproate, norethindrone, norethindrone acetate, norethynodrel, allylestrenol, 19-nortestosterone, lynoestrenol, quingestanol acetate, medrogestone, norgestrienone, dimethisterone, ethisterone, cyproterone acetate, chlormadinone acetate, megestrol acetate, norgestimate, norgestrel, desogrestrel, trimegestone, gestodene, nomegestrol acetate, progesterone, 5.alpha.-pregnan-3.beta., 20.alpha.-diol sulfate, 5.alpha.-pregnan-3.beta., 20.beta.-diol sulfate, 5.alpha.-pregnan-3.beta.-ol-20-one, 16,5.alpha.-pregnen-3.beta.-ol-20-one, 4-pregnen-20.beta.-ol-3-one-20-sulfate, acetoxypregnenolone, anagestone acetate, cyproterone, dihydrogesterone, fluorogestone acetate, gestadene, hydroxyprogesterone acetate, hydroxymethylprogesterone, hydroxymethyl progesterone acetate, 3-ketodesogestrel, megestrol, melengestrol acetate, norethisterone and derivatives, esters, salts and mixtures thereof.

Suitable hydroxyacids include, but are not limited to agaricic acid, aleuritic acid, allaric acid, altraric acid, arabiraric acid, ascorbic acid, atrolactic acid, benzilic acid, citramalic acid, citric acid, dihydroxytartaric acid, erythraric acid, galactaric acid, galacturonic acid, glucaric acid, glucuronic acid, glyceric acid, glycolic acid, gularic acid, gulonic acid, hydroxypyruvic acid, idaric acid, isocitric acid, lactic acid, lyxaric acid, malic acid, mandelic acid, mannaric acid, methyllactic acid, mucic acid, phenyllacetic acid, pyruvic acid, quinic acid, ribaric acid, ribonic acid, saccharic acid, talaric acid, tartaric acid, tartronic acid, threaric acid, tropic acid, uronic acids, xylaric acid, and derivatives, esters, salts and mixtures thereof.

Suitable keratolytic agents include, but are not limited to N-acetylcysteine, glycolic acid, pyruvic acid, resorcinol, sufur, salicyclic acid, retinoic acids and derivatives, esters, salts and mixtures thereof.

Suitable lactams include, but are not limited to L-galactono-1,4-lactam, L-arabino-1,5-lactam, D-fucono-1,5-lactam, D-glucaro-1,4-lactam, D-glucurono-6,3-lactam, 2,5-tri-O-acetyl-D-glucurono-6,3-lactam, 2-acetamido-2-deoxyglucono-1,5-lactam, 2-acetamido-2-deoxygalactono-1,5-lactam, D-glucaro-1,4:6,3-dilactam, L-idaro-1,5-lactam, 2,3,5,tri-O-acetyl-D-glucaro-1,4-lactam, 2,5-di-O-acetyl-D-glucaro-1,4:6,3-dilactam, D-glucaro-1,5-lactam methyl ester, 2-propionoamide-2-deoxyglucaro-1,5-lactam and derivatives, esters, salts and mixtures thereof.

Suitable non-steroidal anti-inflammatory agents include, but are not limited to oxicams, piroxicam, isoxicam, tenoxicam, sudoxicam, CP-14,304, salicylates, aspirin, disalcid, benorylate, trilisate, safapryn, solprin, diflunisal, fendosal, acetic acid derivatives, diclofenac, fenclofenac, indomethacin, sulindac, tolmetin, isoxepac, furofenac, tiopinac, zidometacin, acematacin, fentiazac, zomepirac, clindanac, oxepinac, felbinac, ketorolac, fenamates, mefenamic, meclofenamic, flufenamic, niflumic, tolfenamic acids, propionic acid derivatives, ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, tiaprofen, pyrazoles, phenylbutazone, oxyphenbutazone, feprazone, azapropazone, trimethazone and derivatives, esters, salts and mixtures thereof.

Suitable pediculicides include, but are not limited to DDT, lindane, malathion, permethrin and derivatives, esters, salts and mixtures thereof.

Suitable vasodilators include, but are not limited ethyl nicotinate, capsicum extract and derivatives, esters, salts and mixtures thereof.

Suitable wart removers include, but are not limited to imiquimod, podophyllotoxin and derivatives, esters, salts and mixtures thereof.

The exact amount of a given active pharmaceutical ingredient in a pharmaceutical or cosmeceutical wash-off mousse shampoo composition of the present invention is dependent on the need for which the wash-off mousse shampoo composition is intended to be used or the condition the wash-off mousse shampoo composition is formulated to treat, the exact mode of use and the active pharmaceutical ingredient itself.

Generally, the wash-off mousse shampoo composition of the present invention is formulated so as to be in contact with the skin or scalp for sufficient time so as to allow the absorption of a sufficient amount of the active pharmaceutical ingredient therein and/or to allow the active pharmaceutical ingredient to exert its activity.

Thus, the composition is preferably formulated so that the composition is washed out of the hair or skin area to which the composition is applied within 20 minutes, within 15 minutes, within 10 minutes, within 5 minutes or even within 2 minutes of application. As a result, the active pharmaceutical ingredient is washed out along with the dirt and cleansing agent during rinsing.

Accordingly, the concentration of the at least one active pharmaceutical ingredient in the composition of the present embodiments is high enough so as to allow a sufficient amount of the active pharmaceutical ingredient to be absorbed in the skin and/or to exert a substantial activity, before being washed off.

Depending on the nature of the active pharmaceutical ingredient and the other composition components, in some embodiments the concentration of the at least one active pharmaceutical ingredient is at least about 0.01 weight percentages, at least about 0.05 weight percentages, at least about 0.1 weight percentages, at least about 0.5 weight percentages, at least about 1 weight percentage, at least about 2 weight percentages, and even at least about 3 weight percentages, of the total weight of the wash-off mousse shampoo composition.

As used herein throughout, the phrase “weight percentage(s)” describes the weight percentage(s) of an ingredient of the total weight of a composition containing the ingredient. As used herein the term “about” refers to .+−.10%.

Since the wash-off mousse shampoo composition of the present embodiments may include irritant ingredients and further since it may be applied such that it is maintained on the treated area for a certain period of time, as described hereinabove, the composition preferably further includes agents that can reduce or substantially abolish such an irritation.

As used herein, the phrase “acceptable carrier” describes a carrier that does not cause significant irritation to an organism and does not abrogate the biological activity and properties of the applied active ingredient.

As used herein, the phrase “mousse-forming carrier” describes a carrier that is designed to form a mousse, typically as a result of its dispension.

One skilled in the art is well acquainted with various carriers useful for mousse formulations, see for example U.S. Pat. Nos. 6,627,585, 6,589,509, 6,589,518, 6,368,575, 6,395,258, 6,383,472, 6,080,392, 6,045,779, 5,830,438, 5,690,921, 5,681,546, 5,066,481, 4,834,968, 4,900,326, 4,673,569, and especially the U.S. patent application by the same assignee identified by Attorney Docket Number 27246, and references cited therein. A number of preferred formulations of a pharmaceutical or cosmeceutical wash-off mousse shampoo composition of the present invention are discussed hereinfurther.

The mousse-forming carrier used in the context of the present invention typically comprises a propellant. Traditional mousse compositions for application on hair are dispensed from an aerosol container onto damp hair or onto dry hair or can be dispensed from a nonaerosol pump spray having a foam actuator. When dispensed from an aerosol container, the dissolved propellant expands and generates a dense, finely-bubbled foam. The foam is stable when left undisturbed, and may gradually or partially collapse into a liquid when rubbed into the hair. The introduction of air under pressure forms foam when the mousse is applied from a pump spray.

Suitable propellants for use in the context of the present invention include, without limitation, chlorofluorocarbons, hydrofluorocarbons, hydrochlorocarbons, hydrocarbons, dialkylether, alkanes, compressed propellants and the like. Representative examples include but are not limited to nitrous oxide, carbon dioxide, chloropentafluoroethane, dichlorodifluoromethane, diethyl ether, dimethyl ether, nitrogen, propane, iso-butane, n-butane, isopentane, n-pentane, dimethyl ether, trichlorofluoromethane and mixtures thereof. Generally, the propellant makes up between about 3 weight percentages and about 50 weight percentages of the total weight of the wash-off mousse shampoo composition.

A preferred pharmaceutically acceptable mousse-forming carrier useful in formulating a wash-off mousse shampoo composition of the present invention further includes at least one foam-forming agent in addition to the propellant.

As used herein, the phrase “foam-forming agent” describes an agent that further plays a part in the formation of a mousse form. Such agents are also referred to in the art as “foam boosters” and typically include one or more surface-active agents.

As used herein, the phrase “surface-active agent” describes a chemical substance that has a lipophilic group and a hydrophilic group and therefore has the property of modifying the interfacial tension of the liquid in which it is dissolved. This phrase typically includes soaps, detergents, emulsifiers, dispersing agents and wetting agents. Surface-active agents suitable for use in formulating a mousse-forming carrier of the present invention include anionic, nonionic, amphoteric, cationic and zwitterionic surface-active agents. Specific suitable surface-active agents include but are not limited to acyl glutamates, acyl taurates, N-alkoyl sarcosinates, alkyl alkoxy sulfates, alkyl amidopropyl betaines, alkyl arylsulfonates, alkyl amine oxides, alkyl betaines, alkyl carbonates, alkyl carboxyglycinates, alkyl ether carboxylates, alkyl ether phosphates, alkyl ether sulfates, alkyl ether sulfonates, alkyl glyceryl ether sulfates, alkyl glycinates, alkyl phosphates, alkyl succinates, alkyl sulfates, alkyl sulphosuccinates, ammonium alkyl sulphates, ammonium lauryl sulphate, ammonium lauryl sulphosuccinate, ammonium sulfonate, aryl sulfonates, cocamidopropyl betaine, cocodimethyl sulphopropyl betaine, cocomethyl tauride, cocomonoethanolamide, cocodiethanolamide, coco dimethyl carboxymethyl betaine, cocomonoisopropanolamide, disodium laureth sulfosuccinate, dodecylbenzenesulfonate, ethoxylated sorbitan palmitate, ethoxylated sorbitan oleate, ethoxylated sorbitan stearate, fatty acid alkanolamides, fatty acid amino polyoxyethylene sulfates, fatty acids, fatty alcohol ethoxylates, fatty taurides, isothienates, lauryl amine oxide, lauryl betaine, lauryl dimethyl carboxymethyl betaine, lauryl ether carboxylate, lauryl ether sulfate, lauryl glucoside, lauryl sarcosinate, lauryl sulfate, lauryl sulfosuccinate, nonoxynol phosphates, nonyl phenol ethoxylates, olefin sulfonates, octoxynol phosphates, polyethylene glycols, polysorbate 60, sarcosinates, sodium alkyl sulphates, sodium benzene sulfonate, sodium cocamphopropionate, sodium cocoyl isethionate, sodium cumene sulfonate, sodium dodecylbenzene sulphonate, sodium lauroyl isethionate, sodium N-lauryl sarcosinate, sodium laureth sulphate, sodium lauryl sulphate, sodium oleyl succinate, sodium xylene sulfonate, sulfated monoglycerides, sulfobetaines, sulfosuccinates, sultaines, taurates, triethanolamine dodecylbenzene sulphonate, triethanolamine lauryl sulphate, triethanolamine monolauryl phosphate, alkyldimethylbenzyl chloride ammonium salts, alkyldimethylbenzyl bromide ammonium salts, alkyltrimethylbenzyl chloride ammonium salts, alkyltrimethylbenzyl bromide ammonium salts, cetyltrimethylammonium chloride, cetyltrimethylammonium bromide, tetradecyltrimethylammonium chloride, tetradecyltrimethylammonium bromide, alkyldimethyl hydroxyethylammonium chloride, alkyldimethyl hydroxyethyl ammonium bromide, dialkyldimethylammonium chloride, dialkyldimethylammonium bromide, alkylpyridinium salts, lauryl pyridinium chloride, cetyl pyridinium chloride, alkylamidoethyltrimethylammonium ether sulfates, amine oxides, alkylmethylaminoxide, alkylaminoethyldimethylaminoxide and derivatives, esters, salts and mixtures thereof.

The concentration of surface-active agents in a composition of the present invention can range between about 0.1 weight percetange and about 50 weight percentages of the total weight of the wash-off mousse shampoo composition and preferably ranges between about 5 weight percentages and 50 weight percentages of the total weight of the composition, more preferably between about 10 weight percentages and 50 weight percentages of the total weight of the composition, more preferably between about 15 weight percentages and 50 weight percentages of the total weight of the composition, and even more preferably between about 20 weight percentages and 50 weight percentages of the total weight of the composition.

Emulsifiers suitable for use in formulating a mousse-forming carrier of the present invention include but are not limited to sorbitan isostearate, sorbitan oleate, sorbitan sesquioleate, sorbitan trioleate, polyglyceryl-3-diisostearate, polyglycerol esters of oleic/isostearic acid, polyglyceryl-6 hexaricinolate, polyglyceryl-4-oleate, polygylceryl-4 oleate/PEG-8 propylene glycol cocoate, oleamide DEA, sodium glyceryl oleate phosphate, hydrogenated vegetable glycerides phosphate, glyceryl monostearate, diethylaminoethyl alkyl amide phosphate, glyceryl, glycol esters of stearic acid, eicosene copolymer, sorbitan oleate, and derivatives, esters, salts and mixtures thereof.

The concentration of emulsifiers in a wash-off mousse shampoo composition of the present invention is generally between about 0.01 weight percentage and about 10 weight percenatges of the total weight of the wash-off mousse shampoo composition.

The phrase “wetting agent” as used herein refers to a chemical substance that increases the spreading and penetrating properties of a liquid by lowering its surface tension, i.e., the tendency of its molecules to adhere to each other and/or to other substances such as the solid surfaces they wet. Examples of wetting agent in the context of the present invention include, without limitation such substances as ammonium sulphate, 1,3-butylene glycol, glycerin, propylene glycol, pyroglutamic acid salts, triethanolamine sulphate, sodium lauryl sulphate, polysorbate 60, polysorbate 20, polysorbate 40, polysorbate 80, benzalkonium chloride, docosate sodium, poaxamer, polyoxyethylene alkyl ethers, polyoxyethylene castor oil derivatives, polyoxyethylene stearate, sodium lauryl sulfate, and sorbitan esters.

The mousse-forming carrier may further comprise at least one component selected from the group consisting of fatty alcohols, hydrocarbon alcohols and water.

As used herein, the phrase “fatty alcohol” describes a non-aromatic hydrocarbon alcohol having at least ten carbon atoms and no more than one alcohol group. Fatty alcohols suitable for use in formulating a mousse-forming carrier of the present invention include but are not limited to ethoxylated fatty alcohols having between 10 and 22 carbon atoms. When present, the concentration of fatty alcohols in a composition of the present invention is generally between about 0.01 weight percentage and about 20 weight percenatges of the total weight of the wash-off mousse shampoo composition.

As used herein, the phrase “hydrocarbon alcohol” describes a hydrocarbon that is substituted by one or more hydroxyl groups. Hydrocarbon alcohols suitable for use in formulating a mousse-forming carrier of the present invention include but are not limited to alcohols having between 1 and 10 carbon atoms and more preferably between 1 and 6 carbon atoms, especially aliphatic hydrocarbon alcohols. The aliphatic chain is branched or un-branched, saturated or unsaturated, preferably saturated. Example of suitable hydrocarbon alcohols include but are not limited to methanol, ethanol, n-propanol, isopropanol, n-butanol, sec-butanol, isobutanol and t-butanol and mixtures thereof. When present, the concentration of hydrocarbon alcohols in a composition of the present invention is generally between about 0.01 weight percentage and about 15 weight percenatges of the total weight of the wash-off mousse shampoo composition.

The concentration of water in a composition of the present invention is can range between about 0.5 weight percentage and about 95 weight percenatges of the total weight of the wash-off mousse shampoo composition and preferably ranges between about 40 weight percentages and about 90 weight percentages and more preferably between about 70 weight percentages and about 80 weight percentages of the total weight of the composition.

In some embodiments, a wash-off mousse shampoo composition presented herein includes, in addition to a mousse-forming carrier, a cleansing agent and an active pharmaceutical ingredient, one or more additional ingredients. Such additional ingredients may serve to provide an added value to the composition, to increase acceptance, stability, and the like, to provide aesthetic characteristics, to perform additional pharmaceutical, cosmeceutical, or cosmetic functions, or to add other functionalities.

One skilled in the art is well acquainted with the use and combination of various additional ingredients in mousse formulations see, for example, the references cited above. It is important to note that in some cases a specific additional ingredient also serves as a component of the carrier or serves two or more additional functions. For example, in a specific composition ethanol can serve as a preservative, as a viscosity modifier and as a solubilizer. Suitable additional ingredients include antimicrobial agents, antioxidants, antiperspirants, anti-static agents, astringents, buffering agents, bulking agents, chelating agents, colorants, conditioners, deodorants, detoxifiers, diluents, dyes, emollients, fragrances, foam stabilizers, hair conditioners, humectants, occlusive agents, oils, opacifiers and pearling agents (pearlescent aids), penetration enhancers, perfuming agents, permeation enhancers, pH-adjusting agents, preservatives, protectants, skin penetration enhancers, softeners, solubilizers, sun blocking agents, sunless tanning agents, sunscreens, ultraviolet light absorbers, viscosity modifiers and vitamins. In some instances a specific additional ingredient may have more than one activity, function or effect.

Suitable anti-static agents include but are not limited to synthetic quaternized polymers (e.g., polyquaternium-7), quaternized protein or protein-silicon copolymer, quaternized protein hydrolizate (e.g., croquet L), cationic cellulose derivative (e.g., polyquaternium 10), poly(diallyldimethylammonium chloride), tricetyl methyl ammonium chloride and any derivatives and mixtures thereof.

Suitable buffering agents include, but are not limited to citrate buffers, acetic acid/sodium acetate buffers and phosphoric acid/sodium phosphate buffers.

Suitable conditioners include, but are not limited to cationic surface-active agents such as quaternary ammonium hydroxides, tetramethylammonium hydroxide, alkyltrimethylammonium hydroxides, octyltrimethylammonium hydroxide, dodecyltrimethyl ammonium hydroxide, hexadecyltrimethylammonium hydroxide, cetyltrimethylammonium hydroxide, octyldimethylbenzylammonium hydroxide, decyldimethyl-benzylammonium hydroxide, stearyldimethylbenzylammonium hydroxide, didodecyldimethylammonium hydroxide, dioctadecyldimethylammonium hydroxide, tallow trimethylammonium hydroxide, cocotrimethylammonium hydroxide, cetylpyridinium hydroxide, polyalkylaryl siloxanes, polyalkyl siloxanes, polydimethyl siloxanes, polydiethyl siloxanes, polydimethyl siloxane polymers, polydimethyl siloxane/diphenyl/methylvinylsiloxane copolymers, polydimethylsiloxane/methylvinylsiloxane copolymers and derivatives and mixtures thereof.

Suitable emollients include, but are not limited to mineral oil, lanolin oil, coconut oil, cocoa butter, olive oil, aloe vera extract, jojoba oil, castor oil, fatty acids, fatty alcohols, diisopropyl adipate, hydroxybenzoate esters, benzoic acid esters of C.sub.9 to C.sub.15 alcohols, isononyl iso-nonanoate, silicone oils, polyethers, C.sub.12 to C.sub.15 alkyl benzoates, oleic acid, stearic fatty acid, cetyl alcohols, hexadecyl alcohol, dimethyl polysiloxane, polyoxypropylene cetyl ether, polyoxypropylene butyl ether, and derivatives, esters, salts and mixtures thereof.

Suitable foam stabilizers include, but not limited to capramide DEA, cellulose gum, cocamide DEA, cocamide MEA, cocamidopropyl betaine, cocamidopropylamine oxide, cocamine oxide, coco-betaine, dihydroxyethyl C12-15 alkoxypropylamine oxide, dimethicone copolyol, fatty alkylolamide condensate, hydrolyzed animal protein, hydroxyethyl methylcellulose, hydroxyethylcellulose, hydroxypropyl methylcellulose, hydroxypropylcellulose, isostearamide DEA, lauramide DEA, lauramidopropyl betaine, lauramine oxide, laureth-3, lauryl alcohol, lauryl betaine, lecithin, linoleamide DEA, methylcellulose, myristamide DEA, myristamine oxide, myristoyl hydrolyzed animal protein, oleamide MIPA, oleyl betaine, palm kernelamide dea, peg-6 cocamide, PVP, quaternium-41, sodium methyl cocoyl taurate, stearamide DEA, stearamine oxide, tallow amidopropylamine oxide, oleamide DEA, and derivatives, esters, salts and mixtures thereof.

Suitable fragrances include, but are not limited to menthol, eugenol, phenoxyethanol, isopropyl palmitate, isopropyl myristate, benzyl salicylate, phenylethyl salicylate, thymol, isoamyl salicylate, phenylethyl salicylate, benzoic acid, benzyl benzoate, methyl salicylate, phenol, oleic acid, caproic acid, carbaryl, balm mint extract, carrot oil, chamomile extract, dipentene, eucalyptus oil, fennel extract, geranium oil, juniper tar, lemon extract, matricaria extract, mentol, oil of Italian mandarine, pine tar, rosemary extract, sage extract, sandalwood, thym extract, FRAGRANCE 3949-5, FRAGRANCE 520A FRAGRANCE 91-122, FRAGRANCE HERBAL 10396, FRAGRANCE UNGERER HONEYSUCKLE K 2771, and derivatives, esters, salts and mixtures thereof.

Suitable humectants include, but are not limited to acetamide monoethanolamine, alkoxylated glucose, allantoin, allantoin acetyl methionine, aloe, aloe vera, aloe vera gel, ammonium glycolate, ammonium lactate, butylene glycol, calcium chloride, glycerin, glycine, glycolate salts, glycolic acid, hexanetriol, hexylene glycol, a hexylene glycol derivative, honey, hyaluronic acid, hydrolyzed animal protein, hydrolyzed milk protein, inositol, keratin amino acids, keratin polypeptides, lactamide monoethanolamine, lactate salts, lactic acid, lactose, polyethylene glycol, polyhydroxy alcohol, propylene glycol, quaternary alkyl ammonium glycolate, quaternary alkyl ammonium lactate, sorbitol, a starch, a starch derivative, a sugar, a sugar derivative, urazole, urea, and derivatives, esters, salts and mixtures thereof.

Suitable pH-adjusting agents include, but are not limited to adipic acid, calcium hydroxide, citric acid, glycine, hydrochloric acid, lactic acid, magnesium aluminometasilicates, phosphoric acid, sodium carbonate, sodium citrate, sodium hydroxide, sorbic acid, succinic acid, tartaric acid, and derivatives, salts and mixtures thereof.

Suitable preservatives include but are not limited to C12 to C15 alkyl benzoates, alkyl p-hydroxybenzoates, aloe vera extract, ascorbic acid, benzalkonium chloride, benzoic acid, benzoic acid esters of C9 to C15 alcohols, butylated hydroxytoluene, diazolidinyl urea, DMDM hydantoin, ethanol, hydroxybenzoate esters, iodopropynyl butylcarbamate, methylparaben, polyoxypropylene butyl ether, polyoxypropylene cetyl ether, potassium sorbate, sodium benzoate, sodium bisulfite, sorbic acid, esters, salts and mixtures thereof.

Suitable antioxidants include, but are not limited to butylated hydroxyanisole (BHA), butylated hydroxytoluene (BHT), lecithin, potassium sorbate, propylene glycol, sodium bisulfate, sodium sulfite, sorbic acid, tocopherol, wheat germ and wheat germ oil.

Suitable antimicrobial agents include, but are not limited to benzalkonium chloride, benzoic acid, benzyl alcohol, boric acid, 2-bromo-2-nitropropane-1,3-diol, butylene glycol, captan, chloromethyl isothiazolinone, chloroxylenol, dehydroacetic acid, dimethoxane, disodium monoundecylenamido MEA sulfosuccinate, DMDM hydantoin, eucalyptus oil, formaldehyde, glutaral, isopropyl alcohol, isopropyl cresols, imidazolidinyl urea, MDM hydantoin, methyl Isothiazolinone, methylparaben, myristalkonium chloride, phenoxyethanol, potassium sorbate, propylene glycol, propylparaben, quaternium-14, quaternium-15, resorcinol, low alcohols, SD alcohol 23-A, SD alcohol 38-B, SD alcohol 3-A, SD alcohol 40, SD alcohol 40-B, sodium dehydroacetate, sodium benzoate, sodium bisulfate, sodium salicylate, sodium sulfite, sorbic acid, zinc phenolsulfonate, zinc pyrithione, potassium undecylenoyl hydrolyzed animal protein and salicylic acid.

Suitable detoxifiers include, but are not limited to acetamide MEA, allantoin, aloe, cholesterol, cocamidopropyl betaine, cocamidopropylamine oxide, coco-betaine, disodium hlouolauramido MEA sulfosuccinate, disodium monococamido MIPA sulfosuccmate, disodium monolaureth sulfosuccinate, disodium monooleamido MEA sulfosuccinate, disodium monooleamido PEG-2 sulfosuccinate, disodium monooleamido sulfosuccinate, laneth-16, laneth-10 acetate, laneth-9 acetate, lauramidopropyl betaine, lauramine oxide, lauroyl sarcosine, lauryl betaine, myristamine oxide, oleyl betaine, PEG-10 sorbitan laurate, PEG-40 lanolin, peg-44 sorbitan laurate, PEG-75 lanolin, PEG-85 lanolin, polysorbate 20, polysorbate 40, polysorbate 80, potassium coco-hydrolyzed animal protein, PPG-12-PEG-50-lanolin, PVP, sodium cocoyl sarcosinate, sodium laureth-12 sulfate, sodium lauroyl sarcosinate, sodium/TEA-lauroyl hydrolyzed animal protein, stearamine oxide, TEA-coco-hydrolyzed animal protein, TEA-oleoyl sarcosinate, cocamidopropyl sultaine, cocamine oxide, cocoyl sarcosine and disodium monococamidosulfo succinate.

Suitable astringents include, but are not limited to apple juice, birch leaf extract, birch sap, calcium chloride, cucumber juice, cucumber oil, cupric acetate, fennel extract, horsetail extract, isopropyl alcohol, lemon extract, lemon juice, nettle extract, rosemary extract, witch hazel, witch hazel distillate, witch hazel extract, yarrow extract, zinc acetate, zinc oxide, zinc phenolsulfonate and sage extract.

Suitable skin penetration enhancers include but are not limited to acetone, acyl lactylates, acyl peptides, acylsarcosinates, alkanolamine salts of fatty acids, alkyl benzene sulphonates, alkyl ether sulphates, alkyl sulphates, anionic surface-active agents, benzyl benzoate, benzyl salicylate, butan-1,4-diol, butyl benzoate, butyl laurate, butyl myristate, butyl stearate, cationic surface-active agents, citric acid, cocoamidopropylbetaine, decyl methyl sulfoxide, decyl oleate, dibutyl azelate, dibutyl phthalate, dibenzyl sebacate, dibutyl sebacate, dibutyl suberate, dibutyl succinate, dicapryl adipate, didecyl phthalate, diethylene glycol, diethyl sebacate, diethyl-m-toluamide, di(2-hydroxypropyl)ether, diisopropyl adipate, diisopropyl sebacate, N,N-dimethyl acetamide, dimethyl azelate, N,N-dimethyl formamide, 1,5-dimethyl-2pyrrolidone, dimethyl sebacate, dimethyl sulphoxide, dioctyl adipate, dioctyl azelate, dioctyl sebacate, 1,4 dioxane, 1-dodecylazacyloheptan-2-one, dodecyl dimethyl amine oxides, ethyl caprate, ethyl caproate, ethyl caprylate, 2-ethyl-hexyl pelargonate, ethyl-2-hydroxypropanoate, ethyl alcohol, ethyl laurate, ethyl myristate, 1-ethyl-2-pyrrolidone, ethyl salicylate, hexyl laurate, 2-hydroxyoctanoic acid, 2-hydroxypropanoic acid, 2-hydroxypropionic acid, isethionates, isopropyl isostearate, isopropyl palmitate, guar hydroxypropyltrimonium chloride, hexan-2,5-diol, khellin, lamepons, lauryl alcohol, maypons, metal salts of fatty acids, methyl nicotinate, 2-methyl propan-2-ol, 1-methyl-2-pyrrolidone, 5-methyl-2-pyrrolidone, methyl taurides, miranol, nonionic surface-active agents, octyl alcohol, octylphenoxy polyethoxyethanol, oleic acid, oleic ethanolamide, pleyl alcohol, pentan-2,4-diol, phenoxyethanol, phosphatidyl choline, phosphine oxides, polyalkoxylated ether glycollates, poly(diallylpiperidinium chloride), poly(dipropyldiallylammonium chloride), polyglycerol esters, polyoxyethylene lauryl ether, polyoxy:polyoxyethylene stearate, polyoxypropylene 15 stearyl ether, poly(vinyl pyridinium chloride), propan-1-ol, propan-2-ol, propylene glycol dipelargonate, pyroglutamic acids, 2-pyrrolidone, pyruvic acids, Quaternium 5, Quaternium 18, Quaternium 19, Quaternium 23, Quaternium 31, Quaternium 40, Quaternium 57, quartenary amine salts, quaternised poly(dimethylaminoethylmethacrylate), quaternised poly(vinyl alcohol), sapamin hydrochloride, sodium cocaminopropionate, sodium dioctyl sulphonsuccinate, sodium laurate, sodium lauryl ether sulphate, sodium lauryl sulphate, sugar esters, sulphosuccinate, tetrahydrofuran, tetrahydrofurfural alcohol, transcutol, triethanolamine dodecyl benzene sulphonate, triethanolamine oleate, urea, water esters, salts and mixtures thereof.

Suitable solubilizers include, but are not limited to, propylene glycol, 1,3-propylene diol, polyethylene glycol, ethanol, propanol, glycerine, dimethyl sulphoxide, dimethyl acetamide, dimethyl formamide, hexylene glycol, propylene carbonate, and derivatives, salts and mixtures thereof.

Suitable sunscreens and ultraviolet light absorbers include, but are not limited to benzophenone-2, benzophenone-3, benzophenone-4, benzophenone-6, benzophenone-8, benzophenone-9, benzophenone-11, benzophenone-12, drometrizole, homosalate, methyl anthranilate, octocrylene, octyl methoxycinnamate, octyl salicylate, uric acid and derivatives, esters, salts and mixtures thereof.

Suitable viscosity modifiers include, but are not limited to carbomer, polyethylene glycol, polypropylene glycol, sodium xylene sulphonate, sodium toluene sulphonate, urea, acacia, alcohol, ammonium laureth sulfate, ammonium myreth sulfate, ammonium pareth-25 sulfate, amphoteric-12, amphoteric-7, bentonite, butylene glycol, carbomer-934, carbomer-941, cationic polymers, cellulose gum, hydroxyethylcellulose, methylcellulose, hydroxyethyl methylcellulose, hydroxypropyl methylcellulose, cetyl alcohol, cocamide DEA, cocamide MEA, cocamidopropyl betaine, cocamidopropyl sultaine, cocamidopropylamine oxide, cocamine oxide, coco-betaine, DEA-laureth sulfate, dihydroxyethyl C12-C15 alkoxypropylamine oxide, dimethyl octynediol, emulsifying wax, ethoxydiglycol, ethyl hexanediol, fatty alkylolamide condensate, glyceryl stearate, guar hydroxypropyltrimonium chloride, hexylene glycol, hydroxyethyl methylcellulose, hydroxyethyl stearamide-mtpa, hydroxypropyl methylcellulose, hydroxypropylcellulose, isopropyl alcohol, isostearamide DEA, laneth-16, lanolin, lauramide DEA, lauramidopropyl betaine, lauramine oxide, laureth-3, lauroyl sarcosine, lauryl betaine, lecithin, linoleamide DEA, magnesium aluminum silicate, methylcellulose, montmorillonite, myristamide DEA, myristamine oxide, oleamide MIPA, oleic acid, oleth-10 phosphate, oleyl betaine, palm kernelamide dea, PEG-14M, PEG-150 distearate, PEG-150 stearate, PEG-16 hydrogenated castor oil, PEG-5M, PEG-8 dilaurate, PEG-8 distearate, PEG-8 laurate, petrolatum, poloxamer 101, poloxamer 182, poloxamer 188, poloxamer 238, potassium stearate, propylene glycol, PVP, quaternium-19, quaternium-41, SD alcohol 23-A, SD alcohol 38-B, SD alcohol 3-A, SD alcohol 40, SD alcohol 40-B, sodium cetyl sulfate, sodium laureth sulfate, sodium myreth sulfate, sodium stearate, sodium xylene sulfonate, sorbitan laurate, stearamide DEA, stearamide MEA-stearate, stearamine oxide, stearic acid, stearyl alcohol, tallow amidopropylamine oxide, TEA-oleoyl sarcosinate and mixtures thereof. Suitable opacifiers and pearling agents include, but are not limited to bentonite, cetyl alcohol, glyceryl stearate, glycol distearate, glycol stearate, hydroxyethyl stearamide-mtpa, lanolin, latex opacifier, magnesium aluminum silicate, magnesium carbonate, mica, montmorillonite, myristic acid, PEG-8 distearate, sodium cetyl sulfate, sodium octoxynol-3 sulfonate, sodium stearate, sodium styrene/acrylates/divinylbenzene copolymer, sodium styrene/peg-10 maleate/nonoxynol-10 maleate/acrylate copolymer, sodium tallow sulfate, stearamide DEA, stearamide MEA-stearate, stearic acid, stearyl alcohol, styrene/acrylate copolymer, talc, tocopherol and zinc oxide.

Other ingredients, selected for their folkloric value, may be added to the formulation of the wash-off mousse shampoo composition of the present inventions such as but not limited to acacia, allantoin biotin, allantoin calcium pantothenate, apple juice, apricot juice, autolyzed yeast, avocado oil, balm mint extract, balsam, balsam canada, balsam oregon, balsam tolu, beer, birch leaf extract, birch sap, carotene, carrot juice, carrot oil, chamomile extract, cholecalciferol, cholesterol, clover blossom extract, coconut acid, coconut oil, collagen, corn oil, cucumber juice, cucumber oil, cupric acetate, dna, egg, egg powder, ergocalciferol, eucalyptus oil, fennel extract, geranium oil, glycerin, henna, henna extract, herbal extract, honey, horsetail extract, hybrid safflower oil, hydrolyzed animal protein, hydrolyzed milk protein, inositol, jojoba extract, jojoba oil, juniper tar, keratin amino acids, keratin polypeptides, lactic acid, lactose, laneth-16, laneth-9 acetate, lanolin, lemon extract, lemon juice, liquid silk complex, malt extract, matricaria extract, matricaria oil, menthol, milk protein, mink oil, myristoyl hydrolyzed animal protein, nettle extract, niacinamide, nonfat dry milk, nucleic acid, olive oil, panthenol, peach kernel oil, pectin, pine tar, placental extract, potassium cocoate, pyridoxine HCl, resorcinol, ribonucleic acid, rosemary extract, safflower oil, sage extract, sandalwood, sandalwood oil, sesame oil, soya acid, soybean oil, sucrose, sulfated castor oil, sweet almond oil, thiamine HCl, thyme extract, tocopherol, tocopheryl acetate, vegetable oil, wheat germ, wheat germ oil, witch hazel, witch hazel distillate, witch hazel extract, yarrow extract, yogurt, zinc acetate and any combination thereof.

The choice of a propellant or combination of propellants, the exact composition of a mousse-forming carrier, the choice of cleansing agent and which additional components are added to a specific formulation of a pharmaceutical or cosmeceutical mousse of the present invention is dependent on such factors as the nature of the active pharmaceutical ingredient, the desired mode of use of the mousse and other factors.

Guidance for formulating preferred mousse-forming carriers useful in implementing a pharmaceutical or cosmeceutical mousse of the present invention include the mousse-forming carriers of the wash-off mousse shampoo compositions taught in U.S. Pat. Nos. 6,627,585, 6,589,509, 6,589,518, 6,368,575, 6,395,258, 6,383,472, 6,080,392, 6,045,779, 5,830,438, 5,690,921, 5,681,546, 5,066,481, 4,834,968, 4,900,326, 4,673,569, and especially the U.S. patent application by the same assignee identified by Attorney Docket Number 27246, and references cited therein. Further preferred formulations of wash-off mousse shampoo compositions of the present invention are described in the Examples below.

The wash-off mousse shampoo composition of the present invention is formulated to deliver an active pharmaceutical ingredient to a skin area of the body and particularly the scalp, so as to treat a medical condition that affects such skin areas.

The wash-off mousse shampoo composition of the present invention is advantageously formulated to deliver an active pharmaceutical ingredient to hirsute skin areas.

Such hirsute areas include, for example, the scalp, the armpit, the loins, the genital areas and in some cases also the arms, the back, the legs and others.

It is therefore preferred that a wash-off mousse shampoo composition of the present invention be packaged in a packaging material and identified in print, in or on the packaging material, for use for a need selected from the group consisting of curing a condition, treating a condition, preventing a condition, treating symptoms of a condition, curing symptoms of a condition, ameliorating symptoms of a condition, treating effects of a condition, ameliorating effects of a condition, and preventing results of a condition. A specific condition and specific use is dependent on the exact formulation of a specific wash-off mousse shampoo composition, especially the nature and amount of the one or more active pharmaceutical ingredients therein.

Typical conditions for which a wash-off mousse shampoo composition of the present invention is formulated are medical conditions and cosmeceutical conditions, especially skin and/or scalp diseases or disorders.

Such skin and/or scalp diseases or disorders include, but are not limited to, acne rosacea, actinic keratoses, actinic porokeratosis, acute inflammatory diseases, age spots, allergic contact dermatitis, alopecia, asteatotic eczema, atopic dermatitis, atopic eczema, bacterial infection, BCC, Bowen's disease, burns, chronic hypertrophic lichen planus, chronic superficial scaling, contact dermatitis, cradle cap, cutaneous T-cell lymphoma, cystic acne, dandruff, Darier's disease, dermatitis, dermatitis herpetiformis, dermatosis, discoid eczema, discoid lupus erythematosus, dry skin, eczema, erythrasma, exfoliative keratolysis, folliculitis, fungal infection, juvenile plantar dermatosis, granuloma annulare, Grover's disease, hair thinning, ichthyosiform dermatoses, ichthyosis, impetigo, infantile eczema, infection, intertrigo, keratosis, keloid scarsm lichen simplex chronicus, lichen planus, lichen striatus, lupus erythematosus, neurodermatitis, palmar hyperkeratosis, palmoplantar psoriasis, papular urticaria, parapsoriasis, pediculosis, pellagra, perifolliculitis, pigmented skin, lesions, pityriasis alba, pityriasis lichenoides, pityriasis rosea, pityriasis rubra pilaris, pityriasis versicolor, plantar hyperkeratosis, neurodermatitis, pruritis, psoriasis, Reiter's syndrome, rosacea, seborrhoeic dermatitis, subacute cutaneous lupus erythematosus, tinea capitis, superficial BCC, warts, wound, wrinkles and yeast infections (especially of Malassezia ovalis, Malassezia furfur, Pityrosporium orbiculare and Pityrosporium ovale).

The teachings of the present invention also provide a method of treatment, the method being substantially effected by (a) administering (e.g., by topically applying) to a skin and/or scalp area (wet or dry, depending on the embodiment) of a mammal (non-human or human) in need thereof, a therapeutically or cosmeceutically effective amount of at least one active pharmaceutical ingredient simultaneously with the administration of a cleansing agent in a mousse form; (b) subsequent to the administering, waiting a period of time; and (c) subsequent to the waiting, rinsing the area. During the waiting period the skin area is cleaned and the active pharmaceutical ingredient acts or is absorbed into the skin and/or scalp. The time can be short (e.g. the usual time for applying a shampoo, spreading and rinsing) or may be longer. Generally, due to the irritating nature of cleansing agents, the hair must typically be rinsed within 20 minutes, within 5 minutes or even within 5 minutes of application of the cleansing agent and the active pharmaceutical ingredient.

By need is meant a need selected from the group consisting of curing a condition, treating a condition, preventing a condition, treating symptoms of a condition, curing symptoms of a condition, ameliorating symptoms of a condition, treating effects of a condition, ameliorating effects of a condition, and preventing the results of a condition. Conditions include medical conditions and cosmeceutical conditions, such as skin and/or scalp diseases and disorders, as is detailed hereinabove.

The specific active pharmaceutical ingredient or ingredients administered is dependent on the need for which the method is implemented and can be selected from the list of active pharmaceutical ingredients delineated hereinabove.

The cleansing agent administered is preferably selected from the group consisting cleansers, detergents and soaps, as is detailed hereinabove.

A prophylactically, therapeutically, pharmaceutically or cosmeceutically effective amount, as used herein, means an amount of an active pharmaceutical ingredient needed to achieve the desired outcome, which is generally to prevent, alleviate or ameliorate the condition or symptoms of the condition described hereinabove. Determination of the effective amount, and consequently the dose and dose frequency, is within the capability of one skilled in the art, especially in light of the detailed disclosure provided herein. Factors in determining the effective amount vary with severity of the condition as well as such factors as the subject being treated, the severity of the condition, the age, body weight and response of an individual patient and the judgment of the prescribing physician.

In a preferred embodiment of this aspect of the present invention, the active pharmaceutical ingredient and the cleansing agent form a part of a pharmaceutical or cosmeceutical wash-off mousse shampoo composition containing same, such that topically applying these agents simultaneously is effected by topically applying such a composition.

The pharmaceutical or cosmeceutical wash-off mousse shampoo composition preferably further comprises a mousse-forming carrier and more preferably it is the pharmaceutical or cosmeceutical wash-off mousse shampoo composition described in detail hereinabove.

According to this embodiment, the at least one active pharmaceutical ingredient is administered by passing a pharmaceutical or cosmeceutical wash-off mousse shampoo composition containing the at least one active pharmaceutical ingredient, the cleansing agent and a mousse-forming carrier from a first volume (e.g. a vessel) having a first pressure through a passage (e.g. a nozzle) into a second volume (e.g. the open air) having a second pressure, the first pressure being greater than the second pressure, so as to effect foaming of the composition.

When implementing the method of the present invention, it is often desired to apply to the treated area additional ingredients in addition to the active pharmaceutical ingredient and the cleansing agent. Thus, the pharmaceutical or cosmeceutical wash-off mousse shampoo composition used in implementing the method of the present invention is often formulated with additional components.

The teachings of the present invention also provide a process for preparing a pharmaceutical or cosmeceutical wash-off mousse shampoo composition as described above and which is useful in implementing the method of the present invention by (a) obtaining a mixture of a cleansing agent, at least one active pharmaceutical ingredient and a pharmaceutically acceptable mousse-forming carrier, (b) placing the mixture in a pressure-resistant vessel, and (c) sealing the pressure-resistant vessel.

In cases where the mousse-forming carrier comprises a propellant, the propellant is added to the mixture separately and subsequent to its placing in the vessel.

Thus, in an embodiment of the process according this aspect of the present invention, the mousse-forming carrier comprises a propellant and placing the mixture in the vessel is effected by placing such a mixture while not adding the propellant thereto; placing the mixture in a pressure-resistant vessel and fitting the vessel with a seal-valve, (c) placing an amount of at least one propellant in the pressure-resistant vessel, and (d) fitting the seal-valve of the pressure-resistant vessel with an actuator.

The types and specific examples of suitable active pharmaceutical ingredients, cleansing agents, suitable mousse-forming carriers and suitable propellants have been discussed hereinabove.

Obtaining a mixture as described above generally involves making a first solution, a second solution, and, if desired, a third and a forth solution and then combining the solutions, so as to achieve the desired mixture. The various solutions are typically prepared under different conditions and/or in different solvents or carriers. Thus, in one exemplary embodiment, a first solution is prepared by dissolving water-soluble components in water at room temperature, whereby the second solution is prepared by dissolving other components in water while heating. In another exemplary embodiment, a first solution is prepared by dissolving water-soluble components in water, optionally while heating and a second solution is prepared by dissolving water-insoluble components in a water-miscible organic solvent, optionally by heating. In still another exemplary embodiment, a first solution is prepared by dissolving water-soluble components in water at room temperature, a second solution is prepared by dissolving other components in water while heating, and a third solution is prepared by dissolving water-insoluble components in a water-miscible organic solvent, optionally by heating.

Combining the solutions can be effected either at room temperature or while heating.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above description illustrate the invention in a non-limiting fashion.

Generally, the nomenclature used herein and the laboratory procedures utilized in the present invention include chemical and analytical techniques with which on skilled in the art is familiar. Unless otherwise defined, technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

A Wash-Off Mousse Shampoo Containing Clobetasol Propionate—Composition 1:

Clobetasol propionate is a corticosteroid topically used for the treatment of skin conditions including severe cases of psoriasis and eczematous dermatitis, which takes effect by reducing swelling, redness and itching associated with the skin condition. In the example that follows, clobetasol propionate is used in a composition directed at treating skin conditions of the scalp.

The alcoholic phase was prepared by dissolving the clobetasol propionate (0.05% weigh percentage of the total weight of the composition) in the ethyl alcohol (5-15% by weight) serving as a solvent and a preservative, and a fragrance (0.01-5%), and mixing the solution at room temperature until the clobetasol propionate is fully dissolved.

The aqueous phase was prepared by mixing purified water (70-80% by weight) as a base vehicle, trisodium citrate (0.2-0.9% by weight) and citric acid (0.01-2.0% by weight) as a buffer, coconut fatty acid diethanol amide (cocamide DEA, 1-5% by weight) serving as a nonionic surfactant, foam boosting and stabilization agent, viscosity control agent, conditioning agent and a solubilization agent, polysorbate 20 (1-5% by weight) serving as a mild foaming agent, a cleansing agent, an anti-irritant and a solubilizer, polyquaternium 10 (0.1-1.0% by weight) serving as a cationic polymer, conditioner and a viscosity control agent, and sodium lauryl ether sulfate (5-15-% by weight) serving as an anionic surfactant, a foaming agent and a cleansing agent.

The alcoholic phase was dissolved in the aqueous phase and the mixture was allowed to stir at room temperature until clear.

The mixture was poured into an aluminum aerosol spraying canister, a valve was attached to the canister, vacuum was applied to the canister and the valve was thereafter crimpled, thereby sealing the canister. A hydrocarbon propellant mixture was then added and an actuator was assembled on the valve.

Table 1 below presents the list of ingredients in Composition 1.

TABLE 1

| | | Percent by |
|---|---|---|
| | | Weight of |
| Ingredient | Assumed Purpose | Composition |
| Clobetasol | active pharmaceutical | 0.05 |
| propionate | ingredient | |
| Ethanol | solvent and a preservative | 5-15 |
| Fragrance | fragrance | 0.01-5 |
| Water | base vehicle | 70-80 |
| Trisodium citrate | buffer | 0.2-0.9 |
| Citric acid | buffer | 0.01-2.0 |
| Coconut fatty acid | nonionic surfactant, foam | 1-5 |
| diethanol amide | boosting stabilization agent, | |
| (cocamide DEA) | viscosity control agent, | |
| | conditioning agent and a | |
| | solubilization agent | |
| Polysurbate 20 | mild foaming agent, a | 1-5 |
| | cleansing agent, an anti- | |
| | irritant and a solubilizer | |
| Polyquaternium 10 | cationic polymer, | 0.1-1.0 |
| | conditioner and viscosity | |
| | control agent | |
| Sodium lauryl ether | anionic surfactant, a foaming | 5-15 |
| sulfate | agent and a cleansing agent | |
| Hydrocarbon | propellant | 3-7 |
| propellant | | |

A Wash-Off Mousse Shampoo Containing Clobetasol Propionate—Composition 2:

The alcoholic phase was prepared by dissolving the clobetasol propionate (0.05% weigh percentage of the total weight of the composition) in the ethyl alcohol (5-15% by weight) serving as a solvent and a preservative, and a fragrant (0.01-5%), and mixing the solution at room temperature until the clobetasol propionate is fully dissolved.

The aqueous phase was prepared by mixing purified water (70-80% by weight) as a base vehicle, trisodium citrate as a buffer (0.2-0.9% by weight), polysorbate 60 (1-10% by weight) serving as a mild foaming agent, a cleansing agent, an anti-irritant and a solubilizer, polyquaternium 10 (0.2-1% by weight) serving as a cationic polymer, conditioner and a viscosity control agent, and sodium lauryl ether sulfate (5-15% by weight) serving as an anionic surfactant, a foaming agent and a cleansing agent, and adjusting the pH to 5-7 by the addition while stirring of solid citric acid (0.01-2% by weight).

The alcoholic phase was dissolved in the aqueous phase and the mixture was allowed to stir at room temperature until clear.

The mixture was poured into an aluminum aerosol spraying canister, a valve was attached to the canister, vacuum was applied to the canister and the valve was thereafter crimpled, thereby sealing the canister. A hydrocarbon propellant mixture was then added and an actuator was assembled on the valve.

Table 2 below presents the list of ingredients in Composition 2.

TABLE 2

| | | Percent by |
|---|---|---|
| | | Weight of |
| Ingredient | Assumed Purpose | Composition |
| Clobetasol | active pharmaceutical | 0.05 |
| propionate | ingredient | |
| Ethanol | solvent and a preservative | 5-15 |
| Fragrance | fragrance | 0.01-5 |
| Water | base vehicle | 70-80 |
| Trisodium citrate | buffer | 0.2-0.9 |
| Polysorbate 60 | mild foaming agent, a | 1-10 |
| | cleansing agent, an anti- | |
| | irritant and a solubilizer | |
| Polyquaternium 10 | cationic polymer, | 0.2-1 |
| | conditioner and viscosity | |
| | control agent | |
| Sodium lauryl ether | anionic surfactant, a foaming | 5-15 |
| sulfate | agent and a cleansing agent | |
| Citric acid | buffer | 0.01-2.0 |
| Hydrocarbon | propellant | 3-7 |
| propellant | | |

A Wash-Off Mousse Shampoo Containing Clobetasol Propionate—Composition 3:

The alcoholic phase was prepared by dissolving the clobetasol propionate (0.05% weigh percentage of the total weight of the composition) in the ethyl alcohol (5-15% by weight) serving as a solvent and a preservative, and a fragrant (0.01-4%), and mixing the solution at room temperature until the clobetasol propionate is fully dissolved.

The aqueous phase was prepared by mixing purified water (70-80% by weight) as a base vehicle, trisodium citrate (0.2-0.9% by weight) serving as a buffer, lauramide DEA (1-10% by weight) serving as a nonionic surfactant, foam boosting and stabilization agent, viscosity control agent, conditioning agent and a solubilization agent, polysorbate 20 (2-5% by weight) serving as a mild foaming agent, a cleansing agent, an anti-irritant and a solubilizer, polyquaternium 10 (0.2-1% by weight) serving as a cationic polymer, conditioner and a viscosity control agent, and sodium lauryl ether sulfate (5-15% by weight) serving as an anionic surfactant, a foaming agent and a cleansing agent, and adjusting the pH to 5-7 by the addition while stirring of solid citric acid (0.01-2% by weight). The resulting mixture was heated to 60.degree. C. and stirred until clear.

The alcoholic phase was dissolved in the cooled aqueous phase and the mixture was allowed to stir at room temperature until clear.

The mixture was poured into an aluminum aerosol spraying canister, a valve was attached to the canister, vacuum was applied to the canister and the valve was thereafter crimpled, thereby sealing the canister. A hydrocarbon propellant mixture was then added and an actuator was assembled on the valve.

Table 3 below presents the list of ingredients in Composition 3.

TABLE 3

| | | Percent by |
|---|---|---|
| | | Weight of |
| Ingredient | Assumed Purpose | Composition |
| Clobetasol | active pharmaceutical | 0.05 |
| propionate | ingredient | |
| Ethanol | solvent and a preservative | 5-15 |
| Fragrance | fragrance | 0.01-5 |
| Water | base vehicle | 70-80 |
| Trisodium citrate | buffer | 0.2-0.9 |
| Lauramide DEA | nonionic surfactant, foam | 1-10 |
| | boosting and stabilization | |
| | agent, viscosity control | |
| | agent, conditioning agent | |
| | and a solubilization agent | |
| Polysorbate 20 | mild foaming agent, a | 2-5 |
| | cleansing agent, an anti- | |
| | irritant and a solubilizer | |
| Polyquaternium 10 | cationic polymer, | 0.2-1 |
| | conditioner and viscosity | |
| | control agent | |
| Sodium lauryl ether | anionic surfactant, a foaming | 5-15 |
| sulfate | agent and a cleansing agent | |
| Citric acid | buffer | 0.01-2.0 |
| Hydrocarbon | propellant | 3-7 |
| propellant | | |

A Wash-Off Mousse Shampoo Containing Clobetasol Propionate—Composition 4:

The alcoholic phase was prepared by dissolving the clobetasol propionate (0.05% weigh percentage of the total weight of the composition) in the ethyl alcohol (8-10% by weight) serving as a solvent and a preservative, and a fragrance (0.01-5%), and mixing the solution at room temperature until the clobetasol propionate is fully dissolved.

The aqueous phase was prepared by mixing purified water (70-80% by weight) as a base vehicle, trisodium citrate (0.3-0.9% by weight) serving as a buffer, polysorbate 60 (2-10% by weight) serving as a mild foaming agent, a cleansing agent, an anti-irritant and a solubilizer, polyquaternium 10 (0.2-5% by weight) serving as a cationic polymer, conditioner and a viscosity control agent, and sodium lauryl ether sulfate (8-10% by weight) serving as an anionic surfactant, a foaming agent and a cleansing agent, and adjusting the pH to 5-7 by the addition while stirring of solid citric acid (0.01-2% by weight). The resulting mixture was heated to 60.degree. C. and stirred until clear.

The alcoholic phase was dissolved in the cooled aqueous phase and the mixture was allowed to stir at room temperature until clear.

The mixture was poured into an aluminum aerosol spraying canister, a valve was attached to the canister, vacuum was applied to the canister and the valve was thereafter crimpled, thereby sealing the canister. A hydrocarbon propellant mixture was then added and an actuator was assembled on the valve.

Table 4 below presents the list of ingredients in Composition 4.

TABLE 4

| | | Percent by |
|---|---|---|
| | | Weight of |
| Ingredient | Assumed Purpose | Composition |
| Clobetasol | active pharmaceutical | 0.05 |
| propionate | ingredient | |
| Ethanol | solvent and a preservative | 8-10 |
| Fragrance | fragrance | 0.01-5 |
| Water | base vehicle | 70-80 |
| Trisodium citrate | buffer | 0.3-0.9 |
| Polysorbate 60 | mild foaming agent, a | 2-10 |
| | cleansing agent, an anti- | |
| | irritant and a solubilizer | |
| Polyquaternium 10 | cationic polymer, | 0.2-5 |
| | conditioner and viscosity | |
| | control agent | |
| Sodium lauryl ether | anionic surfactant, a foaming | 8-10 |
| sulfate | agent and a cleansing agent | |
| Citric acid | buffer | 0.01-2.0 |
| Hydrocarbon | propellant | 3-7 |
| propellant | | |

A Wash-Off Mousse Shampoo Containing Ketoconazole—Composition 5:

Ketoconazole is a broad-spectrum synthetic antifungal agent typically administered either orally or topically to treat a variety of systemic and topical fungal infections such as candida, blastomycosis, histoplasmosis, coccidiomycosis and others and as an anti-dandruff agent.

The entire process of preparation of the wash-off mousse shampoo composition containing ketoconazole was carried out at room temperature either at ambient atmosphere or under nitrogen.

Citric acid (1-4% by weight of the total weight of the finished composition) was dissolved in purified water (70-90% by weight) serving as a base vehicle, and ketoconazole (2.1% by weight) was added thereto and stirred until all the ketoconazole was dissolved.

Sodium lauryl ether sulfate (Standapol ES-2) (5-10% by weight) serving as an anionic surfactant, a foaming agent and a cleansing agent, and disodium laureth sulfosuccinate (Stepan mild SL3) (1-7% by weight) serving as a mild foaming agent, a cleansing agent and a detoxifying agent were added to the acidic aqueous solution and stirred until the resulting mixture was clear.

Sodium hydroxide (0.5-1.5% by weight) was added to adjust the pH to 5-7, and thereafter Germall 115 (imidurea) (0-0.7% by weight) serving as a preservative, edetate di sodium (0-1% by weight) serving as a chelating agent and sodium metabisulfite (0-1% by weight), serving as an antioxidant, were added and the mixture was stirred until clear.

Tween 20 (polysorbate 20) or Tween 60 (polysorbate 60) (0-5% by weight) serving as a mild foaming agent, a cleansing agent, an anti-irritant and a solubilizer, and a fragrance (0-4% by weight) were added thereafter to the mixture and stirred until the mixture was clear.

Coconut fatty acid diethanol amide (cocamide DEA) (1.6-5% by weight) serving as a nonionic surfactant, foam boosting and stabilizing agent, viscosity control agent, conditioner and solubilizer, and crocoate L (Lauradimonium hydroxypropyl hydrolyzed collagen) (0.1-2% by weight) serving as a conditioner and a foam stabilizer, were added to the mixture and the mixture was stirred until clear.

The mixture was poured into an aluminum aerosol spraying canister, a valve was attached to the canister, vacuum was applied to the canister and the valve was thereafter crimpled, thereby sealing the canister. A hydrocarbon propellant mixture was then added and an actuator was assembled on the valve.

Table 5 below presents the list of ingredients in Composition 5.

TABLE 5

| | | Percent by |
|---|---|---|
| | | Weight of |
| Ingredient | Assumed Purpose | Composition |
| Citric acid | buffer | 1-4 |
| Water | base vehicle | 70-90 |
| Ketoconazole | active pharmaceutical | 2.1 |
| | ingredient | |
| Sodium lauryl ether | anionic surfactant, a foaming | 5-10 |
| sulfate (Standapol | agent and a cleansing agent | |
| ES-2) | | |
| Disodium laureth | mild foaming agent, a | 1-7 |
| sulfosuccinate | cleansing agent and | |
| (Stepan mild SL3) | detoxifying agent | |
| Sodium hydroxide | buffer | 0.5-1.5 |
| Germall 115 | preservative | 0-0.7 |
| (imidurea) | | |
| Edetate di sodium | chelating agent | 0-1 |
| Sodium | antioxidant | 0-1 |
| metabisulfite | | |
| Tween 20 | mild foaming agent, | 0-5 |
| (polysorbate 20) or | cleansing agent, anti-irritant | |
| Tween 60 | and solubilizer | |
| (polysorbate 60) | | |
| Fragrance | fragrance | 0.01-5 |
| Coconut fatty acid | nonionic surfactant, foam | 1.6-5 |
| diethanol amide | boosting and stabilizing | |
| (cocamide DEA) | agent, viscosity control | |
| | agent, conditioner and | |
| | solubilizer | |
| Crocoate L | conditioner and foam | 0.1-2 |
| (Lauradimonium | stabilizer | |
| hydroxypropyl | | |
| hydrolyzed collagen) | | |
| Hydrocarbon | propellant | 3-7 |
| propellant | | |

A Wash-Off Mousse Shampoo Containing Ketoconazole—Composition 6:

Sodium lauryl ether sulfate (Standapol ES-2) (6-15% by weight of the total weight of the finished composition) and disodium laureth sulfosuccinate (Stepan mild SL3) (2-7% by weight) were added to half of the total amount of purified water (40-42% by weight), and the resulting mixture was stirred until clear.

Sodium hydroxide (0.5-1.5% by weight) was added to the mixture so as to adjust the pH to 5-7.

Citric acid (1-3% by weight) was dissolved in the remaining amount of the purified water (39-42% by weight), and ketoconazole (2.1% by weight) was added thereto and stirred until all the ketoconazole was dissolved. The resulting solution was thereafter added to the pH adjusted mixture containing the sodium lauryl ether sulfate and the disodium laureth sulfosuccinate, and the combined solutions were stirred until clear.

Germall 115 (imidurea) (0-0.5% by weight), edetate di sodium (0-1% by weight) serving as a chelating agent and sodium metabisulfite (0-1% by weight) serving as an antioxidant, were added thereafter and the mixture was stirred until clear.

Coconut fatty acid diethanol amide (cocamide DEA) (1.6-2% by weight) and crocoate L (Lauradimonium hydroxypropyl hydrolyzed collagen) (0.5-2% by weight) were added thereafter to the mixture and the mixture was stirred until clear.

The mixture was poured into an aluminum aerosol spraying canister, a valve was attached to the canister, vacuum was applied to the canister and the valve was thereafter crimpled, thereby sealing the canister. A hydrocarbon propellant mixture was then added and an actuator was assembled on the valve.

Table 6 below presents the list of ingredients in Composition 6.

TABLE 6

| | | Percent by |
|---|---|---|
| | | Weight of |
| Ingredient | Assumed Purpose | Composition |
| Sodium lauryl ether | anionic surfactant, a foaming | 6-15 |
| sulfate (Standapol | agent and a cleansing agent | |
| ES-2) | | |
| Disodium laureth | mild foaming agent, a | 2-7 |
| sulfosuccinate | cleansing agent and | |
| (Stepan mild SL3) | detoxifying agent | |
| Water | base vehicle | 79-84 |
| Sodium hydroxide | buffer | 0.5-1.5 |
| Citric acid | buffer | 1-3 |
| Ketoconazole | active pharmaceutical | 2.1 |
| | ingredient | |
| Germall 115 | preservative | 0-0.5 |
| (imidurea) | | |
| Edetate di sodium | chelating agent | 0-1 |
| Sodium | antioxidant | 0-1 |
| metabisulfite | | |
| Coconut fatty acid | nonionic surfactant, foam | 1.6-2 |
| diethanol amide | boosting and stabilizing | |
| (cocamide DEA) | agent, viscosity control | |
| | agent, conditioner and | |
| | solubilizer | |
| Crocoate L | conditioner and foam | 0.5-2 |
| (Lauradimonium | stabilizer | |
| hydroxypropyl | | |
| hydrolyzed collagen) | | |
| Hydrocarbon | propellant | 3-7 |
| propellant | | |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described with reference to specific embodiments thereof, many alternatives, modifications and variations will be apparent to those skilled in the art. Accordingly, it is intended that the present invention embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent and patent application was specifically and individually indicated to be incorporated herein by reference. In addition, citation or identification of any reference in this application shall not be construed as an admission that such reference is available as prior art to the present invention.

What is claimed is:

1. A method for treating a disease or disorder of a skin or scalp of a mammal while simultaneously cleansing the skin or scalp, said method comprising the steps of:
(a) administering to said skin or scalp a mousse formed from a composition comprising:
(i) a therapeutically or cosmeceutically effective amount of at least one active pharmaceutical ingredient;
(ii) 10 to 50 percent by weight of a cleansing agent;
(iii) a pharmaceutically acceptable mousse-forming carrier comprising a propellant, said propellant comprising 3 to 50 percent by weight of said composition; and
(iv) water comprising about 40 to about 90 percent by weight of said composition;
(b) waiting a period of time for the active pharmaceutical ingredient to treat said skin or scalp; and
(c) rinsing said skin or scalp with water to remove the mousse.

2. The method of claim 1 wherein said skin or scalp includes a hirsute area.

3. The method of claim 1 further comprising moistening said skin or scalp with water prior to administering said mousse.

4. The method of claim 1 wherein said skin or scalp is dry prior to said administering step.

5. The method of claim 1 wherein administering said mousse includes spreading said mousse over a portion of said skin or scalp to be cleansed and treated.

6. The method of claim 1 wherein said period of time is less than about 20 minutes.

7. The method of claim 1 wherein said period of time is less than about 5 minutes.

8. The method of claim 1 wherein said disease or disorder is selected from the group consisting of acne, acne rosacea, actinic keratoses, actinic porokeratosis, acute inflammatory diseases, age spots, allergic contact dermatitis, alopecia, asteatotic eczema, atopic dermatitis, atopic eczema, bacterial infection, BCC, Bowen's disease, burns, chronic hypertrophic lichen planus, chronic superficial scaling, contact dermatitis, cradle cap, cutaneous T-cell lymphoma, cystic acne, dandruff, Darier's disease, dermatitis, dermatitis herpetiformis, dermatosis, discoid eczema, discoid lupus erythematosus, dry skin, eczema, erythrasma, exfoliative keratolysis, folliculitis, fungal infection, juvenile plantar dermatosis, granuloma annulare, Grover's disease, hair thinning, ichthyosiform dermatoses, ichthyosis, impetigo, infantile eczema, infection, intertrigo, keratosis, keloid scarsm lichen simplex chronicus, lichen planus, lichen striatus, lupus erythematosus, neurodermatitis, palmar hyperkeratosis, palmoplantar psoriasis, papularurticaria, parapsoriasis, pediculosis, pellagra, perifolliculitis, pigmented skin, lesions, pityriasis alba, pityriasis lichenoides, pityriasis rosea, pityriasis rubra pilaris, pityriasis versicolor, plantar hyperkeratosis, neurodermatitis, pruritis, psoriasis, Reiter's syndrome, rosacea, seborrhoeic dermatitis, subacute cutaneous lupus erythematosus, tinea capitis, superficial BCC, warts, wound, wrinkles and yeast infections, Malassezia ovalis infections, Malassezia furfur infections, Pityrosporium orbiculare infections and Pityrosporium ovale infections.

9. The method of claim 1 wherein said active pharmaceutical ingredient is a topical active pharmaceutical ingredient.

10. The method of claim 1 wherein said active pharmaceutical ingredient is selected from the group consisting of active herbal extracts, acaricide, age spots and keratoses removing agents, analgesics, local anesthetics, antiacne agents, antiaging agents, antibacterials, antibiotics, antiburn agents, antidandruff agents, antidepressants, antidermatitis agents, antiedemics, antihistamines, antihelminths, antihyperkeratolyte agents, anti-inflammatory agents, antiirritants, antilipemics, antimicrobials, antimycotics, antioxidants, antipruritics, agents, antipsoriatic agents, antirosacea, antiseborrheic agents, antiseptic, antiswelling agents, antiviral agents, antiyeast agents, astringents, topical cardiovascular agents, chemotherapeutics, corticosteroids, fungicides, hair growth regulators, hormones, hydroxyacids, insecticides, keratolytics, lactams, mitocides, non-steroidal anti-inflammatory agents, pediculicide, progestins, sanatives, scabicide, vasodilators and wart removers.

11. The method of claim 1 wherein said administering step includes passing said composition from a first volume having a first pressure through a passage into a second volume having a second pressure, said first pressure being greater than said second pressure, so as to effect foaming of said composition to form said mousse.

12. The method of claim 1 wherein said cleansing agent comprises an anionic surfactant.

13. The method of claim 12 wherein said cleansing agent further comprises a nonionic surfactant.

14. The method of claim 1 wherein said scalp includes hair and said administering of said mousse includes spreading said mousse through said hair and scalp.

15. The method of claim 1 wherein said pharmaceutically acceptable mousse-forming carrier further comprises an emulsifier.

16. A method for treating a disease or disorder of a skin or scalp of a mammal while simultaneously cleansing the skin or scalp, said method comprising the steps of:
(a) administering to said skin or scalp a mousse formed from a composition comprising:
(i) a therapeutically or cosmeceutically effective amount of corticosteroid;
(ii) 10 to 50 percent by weight of a cleansing agent;
(iii) a pharmaceutically acceptable mousse-forming carrier comprising a propellant, said propellant comprising 3 to 7 percent by weight of said composition; and
(iv) water comprising about 40 to about 90 percent by weight of said composition; and
(b) waiting a period of time for said corticosteroid to treat said skin or scalp; and
(c) rinsing said skin or scalp with water to remove the mousse;
wherein said administering step includes passing said composition from a first volume having a first pressure through a passage into a second volume having a second pressure, said first pressure being greater than said second pressure, so as to effect foaming of said composition into the mousse.

17. The method of claim 16 wherein said corticosteroid includes clobetasol propionate.

18. The method of claim 17 wherein said clobetasol propionate comprises about 0.05 percent by weight of said composition.

19. The method of claim 16 further comprising moistening said skin or scalp with water prior to administering said mousse.

20. The method of claim 16 wherein administering said mousse includes spreading said mousse over a portion of said skin or scalp to be cleansed and treated.

21. The method of claim 16 wherein said period of time is less than about 20 minutes.

22. The method of claim 16 wherein said disease or disorder is selected from the group consisting of psoriasis, eczematous dermatitis, and combinations thereof.
